(19) United States
(12) Patent Application Publication
(10) Pub. No.: US 2002/0024633 A1
(43) Pub. Date: Feb. 28, 2002

(54) Pupil evaluation system

(76) Inventors: Daehoon Kim, Pleasanton, CA (US); Jossif Makartchouk, Moscow (RU); Natalia Vartchenko, Moscow (RU); Konstantin Gankine, Reutov (RU)

Correspondence Address: EDWARD O. KRUESSER, BROBECK PHLEGER & HARRISON, 12390 EL CAMINO REAL, SAN DIEGO, CA, 92130, US

(21) Appl. No.: 09/843,613

(22) Filed: Apr. 25, 2001

Related U.S. Application Data
(63) Continuation-in-part of application No. 09/433,254, filed on Nov. 4, 1999, now Pat. No. 6,247,813.

(30) Foreign Application Priority Data
Apr. 25, 2000 (KR) 10-2000-0022061
Apr. 7, 2000 (KR) PCT/KR00/00324
Apr. 9, 1999 (KR) 10-1999-0012438
Apr. 9, 1999 (KR) 10-1999-0012439
Apr. 9, 1999 (KR) 10-1999-0012440

(51) Int. Cl. A61B 3/14

(52) U.S. Cl. 351/206; 382/117

(57) ABSTRACT

The present invention comprises methods of and systems that evaluate a living being using the pupil and/or autonomous nervous wreath (“ANW”) of the living being. The eye of the living being is subjected to a physical stimulus. Dimensional changes that occur in the pupil and/or ANW of the living being because of the physical stimulus are recorded via a video camera. The images of the pupil and/or ANW are sent to a computer to be analyzed. A pupillogram is constructed of the dimensional changes in the pupil and/or ANW. Based on an analysis of the pupillogram and characteristic parameters of the pupillogram, an evaluation of the living being is made. This evaluation can be used to determine if the living being is intoxicated with a drug, suitable for certain vocational skills or suitable for certain athletic positions.

### RELATED APPLICATIONS

This application is a continuation-in-part (“CIP”) of U.S. patent application Ser. No. 09/433,254, filed Nov. 4, 1999, which is incorporated herein by reference in its entirety; a CIP of Korean Patent Application No. 10-2000-0022061, filed Apr. 25, 2000, which is incorporated herein by reference in its entirety; a CIP of PCT Application No. PCT/KRO0/00324, filed Apr. 7, 2000, which is incorporated herein by reference in its entirety, a CIP of Korean Patent Application No. 10-1999-0012438, filed Apr. 9, 1999, which is incorporated herein by reference in its entirety; a CIP of Korean Patent Application No. 10-1999-0012439, filed Apr. 9, 1999, which is incorporated herein by reference in its entirety; and a CIP of Korean Patent Application No. 10-1999-0012440, filed Apr. 9, 1999, which is incorporated herein by reference in its entirety.

### BACKGROUND

1. Field of Invention

The present invention relates to evaluating parameters of a living being based on dimensional changes of a living being's pupil. More particularly, the present invention relates to a system for detecting drug use in a living being, or for assessing or screening for aptitude, such as for athletics or vocational skills, of a living being based on dimensional changes in a living being's pupil.

2. Description of the Prior Art

Companies and athletic programs currently test living beings to determine whether they have used drugs. Currently those desiring to conduct such tests may test a living being's blood, urine, or hair to test for such drug use. However, testing using these systems has problems. For example, a physical sample of the living being must be removed from the living being. This may be difficult and/or cumbersome. Additionally, it is possible for the living being to “switch” his or her sample, such as a urine sample, with a sample of an individual not using drugs. Moreover, the sample usually must be sent to an off-site laboratory to be tested. These factors add to the cost, time and inconvenience of these tests.

It is also desirable for certain athletes and those performing specific vocational tasks to have specialized skills. For example, it may be more desirable for a living being with exceptional visual or motor skills to perform certain tasks. Also, it may be desirable for a living being with exceptional reaction times or autonomic reflexes to perform certain athletic tasks on a team, to operate specialized machinery, or to be utilized at certain positions in the public service.

Therefore, there is a need for better evaluation of living beings, for example, to determine if the living being has used drugs or is especially qualified to perform certain tasks.

### SUMMARY OF THE INVENTION

An embodiment of the present invention comprises a device for recording dimensional changes in a living being's pupil(s) caused by external stimuli as a function of time. A living being's actual response may be mathematically analyzed to yield numerous parameters which numerically characterize a living being's pupillary response, and thus the present or average state of a living being's autonomic nervous system.

Another embodiment of the present invention comprises a method of evaluating a living being, comprising the steps of: stimulating an eye of a living being using one or more physical stimulus, wherein the eye comprises a pupil; electronically measuring a dimensional change in the pupil in response to the at least one physical stimulus; automatically determining at least one parameter relating to the dimensional change; electronically storing the at least one parameter in a computer; determining at least one normal parameter, wherein the normal parameter comprises information relating to a plurality of other normal living beings that have at least one similar characteristic of the living being, wherein the at least one similar characteristic is age or gender; comparing the at least one parameter with the at least one normal parameter; and evaluating the living being based on the comparison step.

Advantageously, an embodiment of the present invention detects whether a living being has used drugs, or has specific aptitudes, such as for certain athletics or certain vocational skills. Under the present invention, the living being does not have to give a sample, such as a blood or urine sample, to have such tests performed. Additionally, results of the test can be received and evaluated quickly and without the need to send samples to an outside laboratory.

Advantageously further, the results of tests performed according to an embodiment of the present invention are difficult to adulterate or tamper with; the embodiment of the present invention measures an involuntary reflex that is independent of the will of the living being.

Further features and advantages of the invention as well as the structure and operation of various embodiments of the invention are described in detail herein with reference to the accompanying drawings.

### BRIEF DESCRIPTION OF THE DRAWINGS

The present invention will be described with particular embodiments thereof, and references will be made to the drawings in which:

FIG. 1 illustrates a portion of a computer, including a CPU, conventional memory, and communications hardware in which the present invention may be embodied;

FIG. 2 illustrates a front view of an embodiment of the present invention;

FIG. 3 illustrates a rear view of an embodiment of the present invention;

FIG. 4 illustrates an embodiment of the present invention;

FIG. 5 illustrates an image of pupillograms according to an embodiment of the present invention;

FIG. 6 illustrates a pupillogram according to an embodiment of the present invention;

FIG. 7 illustrates a flow chart of an embodiment of the present invention;

FIG. 8 shows an eye according to embodiment of the present invention; and

FIG. 9 shows a block diagram of a camera body according to an embodiment of the present invention.

### DETAILED DESCRIPTION OF AN ILLUSTRATED EMBODIMENT

The present invention evaluates a living being based on dimensional changes in the living being's pupil(s), for example, by determining certain parameters characterizing the response of a living being's pupil(s) to a physical stimulus. An embodiment of the present invention records and analyzes dimensional changes of a living being's pupil(s) in response to a physical stimulus and calculates various parameters characterizing this response; these characteristic parameters include the pupil(s) latency time, time and speed of sympathetic and parasympathetic phases, dilation latency duration, recovery, and other such parameters. These characteristic parameters are compared with the characteristic parameters of normal living beings (other than the living being that is being tested) having similar characteristics to the living being, such as age and gender. The characteristic parameters of the normal living being are obtained using multiple measurements of living beings over various ages and both genders that are known not to be intoxicated with a drug or otherwise mentally impaired. The characteristic parameters of living being's pupil are altered when the living being's autonomic nervous system is impaired or compromised, e.g., by a drug, because the pupil is a sensitive measure of the relative state of the autonomic nervous system of the living being. Thus, changes in these characteristic parameters may be correlated with changes expected if a living being is intoxicated or otherwise impaired. Moreover, these characteristic parameters may also be used to identify living beings with exceptional or desirable autonomic reflexes. “Automatic,” “automatically,” “automated,” or the like, when referring to anything but a physical response of an organ of a living being, as used herein, means to occur without human interaction. For example, this may mean that the event has occurred using a computer that is programmed to perform the event using information that the computer has received, obtained and/or gathered. Operation of some embodiments of the present invention allow for the elimination of substantial human effort at various phases, such phases are described herein as being “automatic,” “automated,” occurring “automatically,” or the like. However, human intervention may allow that such phases be completed manually. When referring to a physical response by an organ of a living being, “automatic,” “automatically,” “automated,” or the like, as used herein, means to occur independent of the will of the living being.

The “autonomic nervous system” (“ANS”) is that part of the nervous system that controls involuntary actions, including the smooth muscles, cardiac muscle, and glands. The “parasympathetic nervous system” is that portion of autonomic nervous system responsible for such involuntary functions as the constriction of the pupils, inter alia. The “sympathetic nervous system” is that portion of autonomic nervous system that governs processes associated with response to alarm, such as acceleration of the heart rate, constriction of the blood vessels, elevation of blood pressure, inter alia.

A “living being,” as used herein, means any animate being that the present invention is testing or evaluating or being used to test or evaluate. For example, a living being may be any person or other animal that is capable of having its eyes evaluated as described herein. The living being preferably has two eyes, but a living being having any number of eyes can be evaluated according to the present invention.

An “operator,” as used herein, means any individual, computer, or other entity that is operating the system that evaluates the pupil of a living being.

A “drug,” as used herein, means any drug, including alcohol, that may impair a living being when used, taken, inhaled, injected, ingested, digested, or in any way inserted into the living being's body, especially in chemical contact with the central nervous system of the living being. When referring to a drug that has been inserted into a living being's body, such reference includes any way in which such drug has been inserted into the living being's body, regardless of whether the drug was inserted voluntarily or involuntarily.

A “normal living being,” as used herein, is a living being that is not drug impaired nor has any known impairments that would affect its ANS.

The term “dimensional change” as used herein means a size change, e.g., a change in the radius of a pupil brought about by a physical stimulus. Such changes may include pupillary constriction (leading to a smaller pupillary radius) or pupil dilation (leading to an increase in pupillary radius). In one embodiment, dimensional changes refer to the distance from a moving pupil border to the center of the iris or pupil.

The term “stimulating the eye with a light source” means that an external light source enters into an eye and impinges on the retinal surface of the eye.

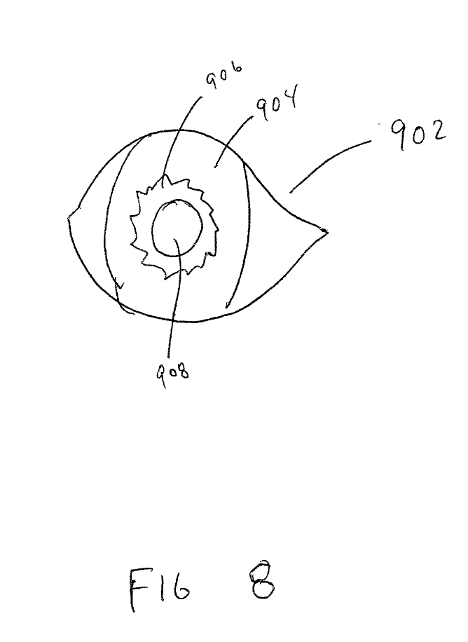

The “autonomous nervous wreath” (“ANW”), is also known as the autonomous wreath, autonomic nerve wreath, iris crown, sympathetic crown, iris fringe, frill and collarette. The ANW may be considered to be an index of the autonomic nervous system. An example of an ANW is shown in FIG. 8, which shows an eye according to embodiment of the present invention. Eye 902 is an eye of human living being, and includes an ANW 906, pupil 908, and iris 904.

The “pupil” of a living being, as used herein, means the adjustable opening at the center of the iris of the living being that allows varying amounts of light to enter the eye. Pupillary constriction and dilation are controlled by the ANS. Anywhere herein that indicates an evaluation of the pupil may also include an evaluation of the ANW. Accordingly, the term pupil, as used herein includes the pupil and/or the ANW.

A “physical stimulus,” “stimulus,” or “stimuli,” as used herein, is one or more sensory stimuli to which a pupil responds, for example, light and other electromagnetic energy, sound, tactile contact, pain, chemical irritant(s), taste, changes in temperature, or the like. As used herein, when something is stated as being stimulated by a physical stimulus, that also includes the case where the something has been stimulated by a physical stimulus for a period of time, and the physical stimulus is then removed.

To access a system in a “secure manner,” as used herein, means to access the system in an exclusive, private manner. Accessing a system in a secure manner may be implemented using an alphanumeric pin code, password, biometric information (e.g., a fingerprint, voice or retinal recognition), knowledge based information (e.g., a mother's maiden name), any combination of the above, or the like. Encryption methods can also be used to provide such access to the system.

A “computer,” as used herein, includes any general-purpose machine that processes data according to a set of instructions that is stored internally either temporarily or permanently, including, but not limited to, a general purpose computer, a workstation, a laptop computer, a personal computer, a set top box, a web access device (such as WEB TVTM (Microsoft Corporation)), cable television, satellite television, broadband network, an electronic viewing or listening device, wireless devices, such as a personal digital assistant (“PDA”), cellular or mobile telephones, an electronic handheld unit for the wireless receipt and/or transmission of data, such as a BLACKBERRY™ (Research In Motion Limited Corporation), or the like.

“Electronic connection,” as used herein, is any electronic connection, including connections via hardwire, Ethernet, token ring, modem, digital subscriber line, cable modem, wireless, radio, satellite, and combinations thereof. Such connections may be implemented using copper wire, fiber optics, radio waves, coherent light, or other media.

A “system of networked computers” or “network,” as used herein, is any system of multiple computers that are directly or indirectly interconnected by any type of electronic connection. The system of networked computers may be the Internet, an intranet, a secure virtual private network (“VPN”), or any other system of computers that are interconnected by electronic connections. As used herein, the term “network” refers to any such system of networked computers, including the Internet.

An embodiment of the present invention is used to determine whether a living being has been impaired by a drug. Alternatively, an embodiment of the present invention is used to evaluate whether the living being is suitable for a certain position, such as a position in the public service, a certain position on an athletic team, a vocational position or the like. Alternatively still, an embodiment of the present invention is used to evaluate whether a living being has received a trauma or other damage to the living being's brain.

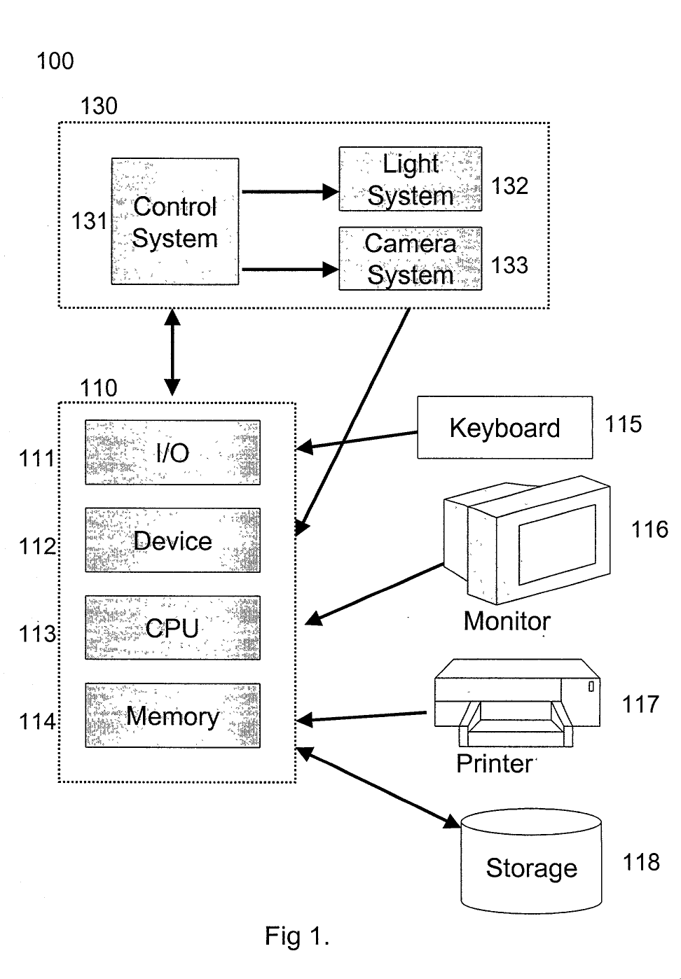

FIG. 1 illustrates a portion of a computer system 100, including a central processing unit (“CPU”) in which the present invention may be embodied. Some of the elements in FIG. 1 shown include a processor 110 having an input/output (“I/O”) section 111, a device 112 (such as a CD-ROM device, disk drive, or the like), a CPU 113, and a memory section 114. The processor 110 is connected to a keyboard 115, a display unit (or monitor) 116, a printer 117, and a storage disk 118 such as a database, hard drive, a CD-ROM or similar unit. The CD-ROM reads a CD-ROM or similar medium, which typically contains programs and data. The processor 110 is also connected to a camera body 130. The camera body 130 contains a control system 131 that controls the camera system 133 and the stimulating device 132 (which may be for example a light system or some other system for emitting a physical stimulus to a living being).

Additionally, a telecommunications system may be connected to the system via a modem or other communications device. This allows the system to connect to a telecommunication network, such that the system may connect to a remote computer system. The present invention may work on a single or plurality of computers, over a system of networked computers, and/or may be locally or remotely operated.

The present invention uses a computer to help measure and to evaluate parameters of a living being's pupil. A system that can measure some of the parameters of a living being's pupil are described in U.S. Pat No. 5,187,506, which is incorporated by reference herein in its entirety. Earlier systems that evaluated a pupil used infrared (“IR”) sensitive plates and/or photodiodes. The area of the pupil was calculated using the number of pixels detected by the plates. However, using IR sensitive plates could result in an error if the living being closed their eyelid(s). Therefore, according to an embodiment of the present invention, such errors do not occur because the pupils are evaluated in real-time using at least one video camera.

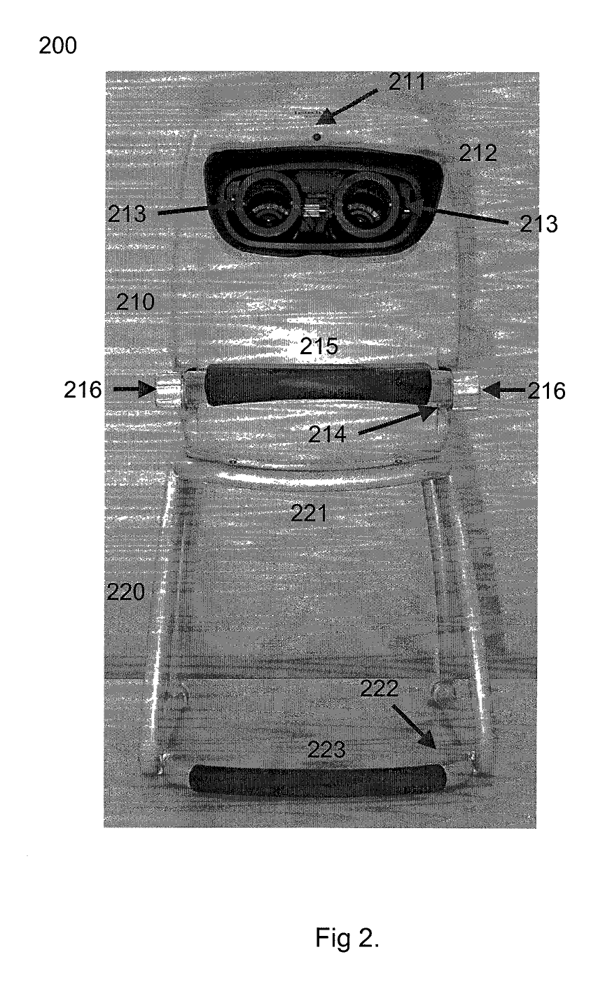

A front view of an embodiment of the present invention is shown in FIG. 2. A front view of camera system 200 is shown. Camera system 200 comprises a viewfinder 212. Viewfinder 212 can be adjusted to allow a living being to use viewfinder 212 comfortably. Two cameras, one behind each lens 213 of viewfinder 212 are also used. These cameras record images of the pupils of the living being. These images are sent to a computer to be evaluated. In another embodiment, one camera is used behind one or both of lenses 213. Body 210 maintains the cameras, lenses 213, and viewfinder 212. Body 210 can be made of plastic, a polymer or some other material, such as a light weight material.

In an embodiment, a living being that is being evaluated by camera system 200 rests his or her chin on chin bar 214. Chin bar 214 is adjustable to fit the head of the living being. Chin bar 214 may be adjusted by using knobs 216. Chin bar 214 comprises an optional pad 215 that is made of foam or some other material that is comfortable on the living being's chin. Alternatively, a forehead bar may be included that allows the living being to rest her or her forehead comfortably when the living being is using viewfinder 212. The optional forehead bar may also be adjustable. Alternatively still, a facial alcove may allow for the living being to use the viewfinder without discomfort of the living being's face, for example, without having the nose of the living being be depressed against body 210. In an alternative embodiment, forehead bar and chin bar 214 are not adjustable. Indicator light 211 illuminates when the camera system is on.

Optional support stand 221 supports and stabilizes camera system 200. The support can be made of aluminum or some other supportive material or alloy. Optional hand grips 220 allow the living being to hold onto support 221 and thus camera system 200 when the living being is using viewfinder 212. Hand grips 220 may be made of foam or some other material to make gripping hand grip 220 more comfortable. Alternatively, hand grips 220 do not have such a covering. Bar 222 comprises a covering, such as a foam covering or some other similar material, that allows the camera system 200 to rest on a surface with a decreased chance that system 200 would slip or accidentally slide on the surface. Bar 222 helps stabilize the camera system 200. Support stand 221 can be folded over on top of body 210 for easy transport or storage. Alternatively, camera system 200 is placed on a table and thus does not need support stand 221.

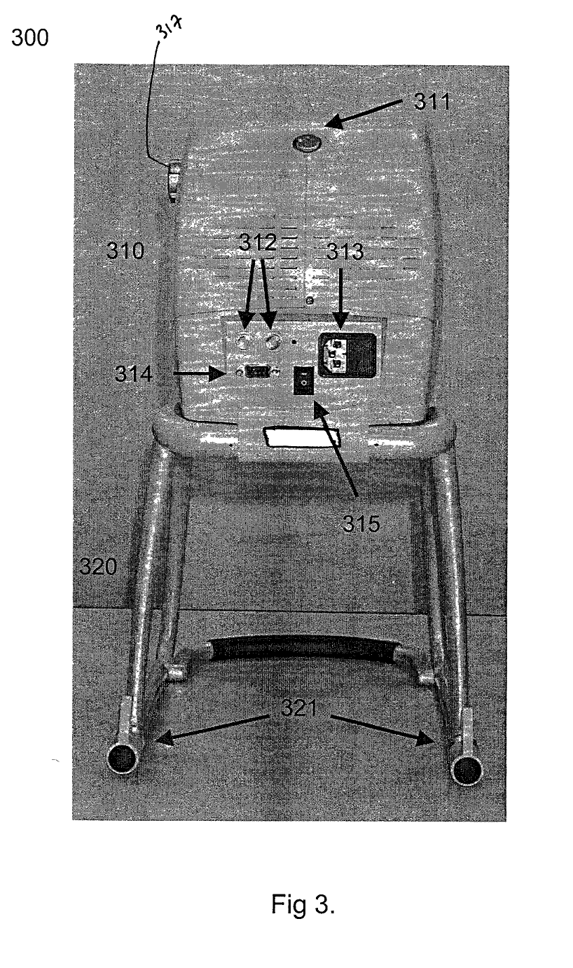

A rear view of an embodiment of the present invention is shown in FIG. 3. A rear view of camera system 300 is shown. Body 310 holds camera system 300 together. Ports 312 are video output ports for each camera in camera system 300, such that the individual cameras in system 300 may be connected to other devices, such as the computer. Alternatively, camera system 300 can also include an input port, such that a signal may be received by the system from some other device, such a control unit.

Control 317 is used by an operator to adjust the viewfinder to fit the living being. For example, control 317 can be adjusted depending on the pupillary distance (“PD”), which is the distance between the pupils of the eyes of the living being. The operator can view the images of the living beings pupils on the computer, and then adjust the viewfinder using control 317 such that the pupils are each centered. Alternatively, if evaluating just one pupil, control 317 is used to center the one pupil on the computer screen. Alternatively, control 317 can be used to control the vertical and horizontal placement of the lenses and viewfinder, such as to adjust the distance the cameras are from the lenses in the viewfinder. Alternatively still, the operator can instruct the living being to adjust control 317 such that the viewfinder is comfortable and the living being's eyes are centered.

Button 311 is an ON/OFF button for the camera system. Additionally, camera system can have additional controls to control the color of the image, the brightness of the image, the size of the image, or the like.

Switch 315 is a power ON/OFF switch that controls whether the camera system 300 is operational. Button 311 and switch 315 can be used such that switch 315 powers down the system, while button 311 is used to indicate that an examination of a living being is about to begin.

Output 314 allows a connection to be established between camera system 300 and a computer. Output 312 may comprise a serial port, parallel port, USB connection, FireWire connection, PS/2 connection, or any other type of data transfer port. Plug connection 313 allows camera system 300 to receive power. The plug connection 313 supports a standard power adapter, such as an AC adapter that converts AC power from a wall outlet into DC power.

Optional support stand 320 allows camera system 300 to be supported as described herein. Ends 321 allow for the length of the support beams of support stand 320 to be adjusted, for additional support. Ends 321 are padded and may be used as a brace for when support stand 320 is placed against a hard surface, such as a wall. By placing support stand 320 against a hard surface, the system is more stable when a living being is viewing the viewfinder.

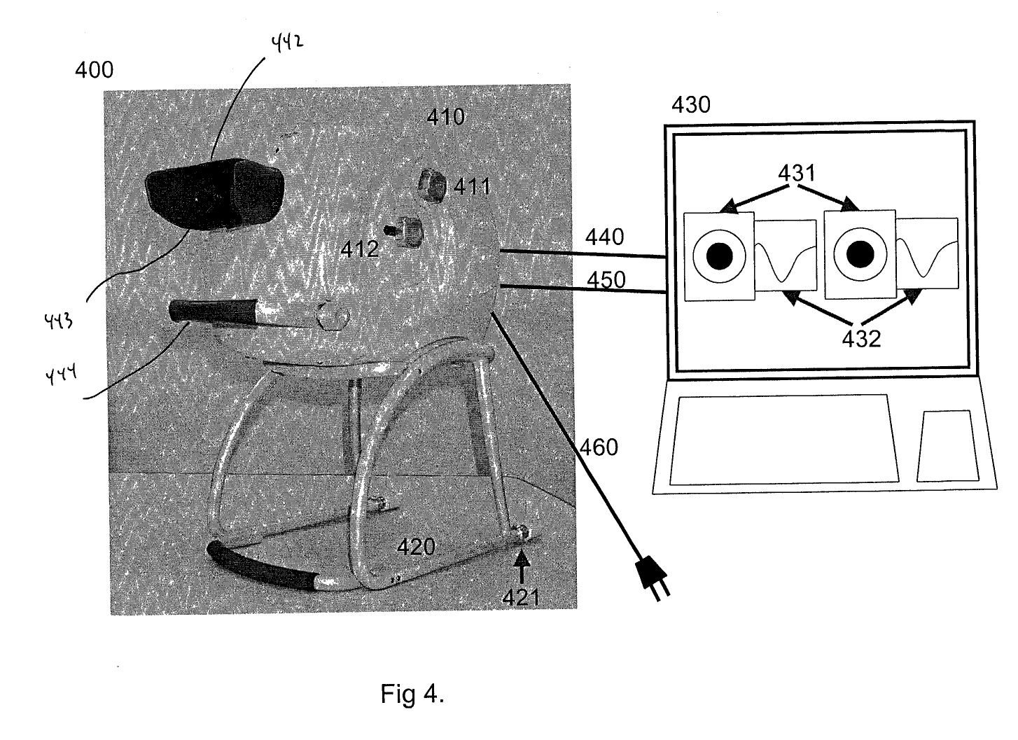

Another embodiment of the present invention is shown in FIG. 4. Camera system 400 comprises a viewfinder 442, lenses 443, body 410, chin bar 444, support stand 420 and ends 421. Control 412 is used to control the distance the camera system is from the lenses 443. Control 412 is also used to adjust for the PD of the living being. Control 411 controls the vertical height of the camera system. Using controls 411 and 412, an operator can center and/or adjust the images 431 of the eyes of the living being on the computer 430.

Computer 430 connects to camera system 400 via connections 440 and 450. Connections 440 and 450 may be any electronic connections. Connections 440 and 450 allow information to flow back and forth between camera system 400 and computer 430. Connection 440 can be information from the system 400 to the computer 430 and connection 450 can be information from each of the cameras in the system 400 to the computer 430. Alternatively, only one connection 440 or 450 is needed for both connections. Power supply 460 provides power to the camera system 400.

When a living being is using viewfinder 442, a camera records images of the living being's eyes, including the pupil, iris, and ANW. The images of the living being's eyes are displayed on the computer as images 431 for the left and right eye. These images may be in real-time, such that the images change as dimensional changes in the pupil(s) occur. Pupillograms 432, for the left and right pupil, are created by computer 420. Pupillograms 432 may be created in real-time, such that the pupillograms 432 change as the size of the pupils change.

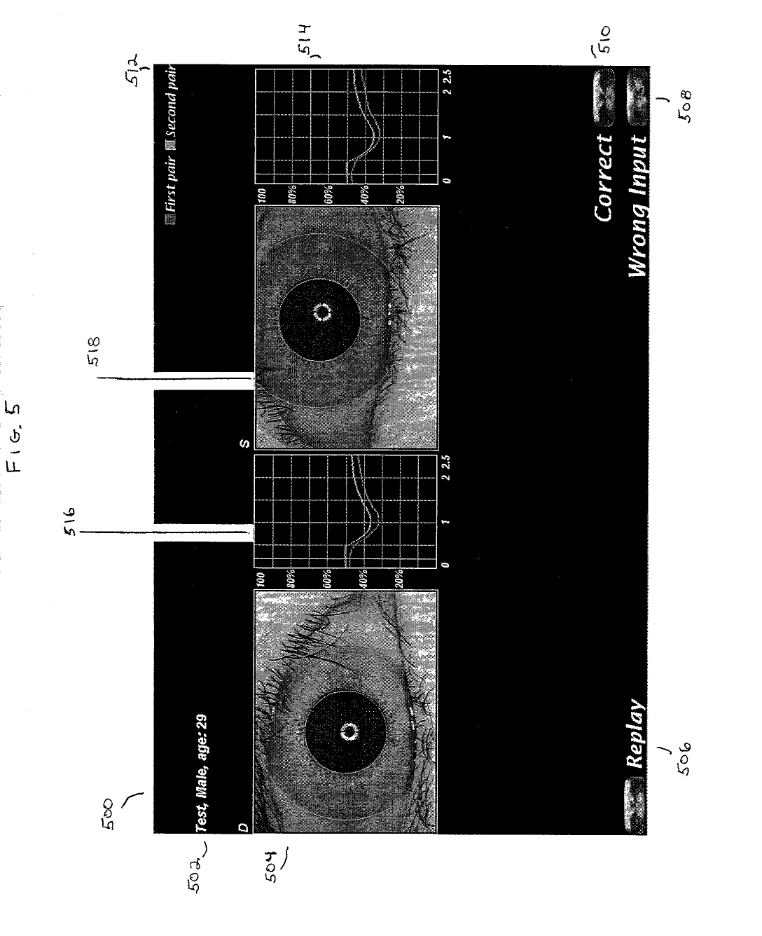

Referring now to FIG. 5, an image of pupillograms according to an embodiment of the present invention is shown. Image 500 is shown on a computer screen. An operator views image 500. Information 502 can include information about the living being that is being tested. For example, information 502 can include the gender, age, and eye color(s) of the living being. Information 502 can also include other information about the living being, such as his or her name, address, weight, height, and the like. The left eye of the living being is viewed in image 504. The right eye of the living being is viewed in image 518. The left and right eye images can be switched. Alternatively, the images of the eyes are automatically centered in the image's box. This can be done by automatically adjusting the camera using the computer, adjusting the image from the camera using the computer, manually adjusting the camera by the operator, or the like. Pupillogram 516 is a pupillogram of the left pupil of the living being after being subjected to a physical stimulus. Pupillogram 514 is a pupillogram of the right pupil of the living being after being subjected to a physical stimulus. On the pupillogram, the y axis is the percentage of the size of the pupil in comparison to the size of the iris and the x axis is time in seconds.

As illustrated in legend 512, pupillograms 516 and 518 show two tests that were run on the living being and the pupillograms that were created for each test. Each test comprises a physical stimulus being applied to the eye of the living being. Alternatively, pupillograms 516 and 518 can show a normal pupillogram superimposed with the pupillogram of the living being's pupil.

Optional buttons on the bottom of screen allow the operator to perform various functions. For example, button 506 allows the test to be replayed again, such that the operator can review the pupils dimensionally change. Button 510 allows the operator to indicate that the test was run successfully. Button 508 allows the operator to indicate that there was an error that occurred during the test, such that the test should be run again.

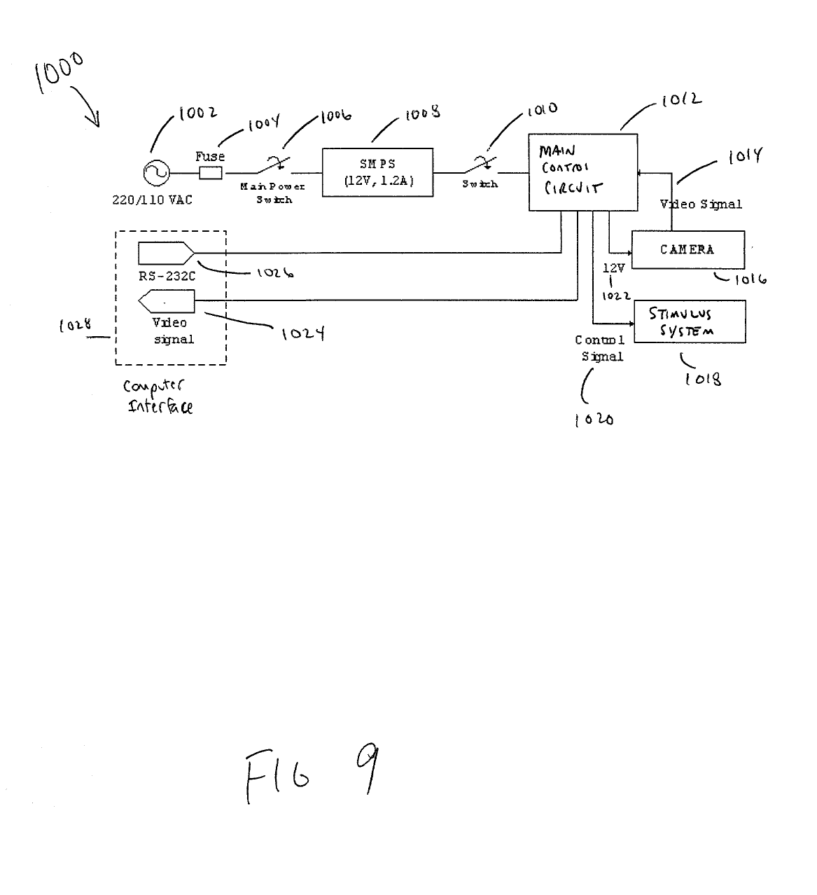

FIG. 9 shows the internal workings of the camera body 1000 according to an embodiment of the present invention. Power 1002 supplies body 1000 through a fuse 1004 to protect against spikes in the power. Main power switch 1006 controls the flow of power to body 1000. A switching mode power supply (“SMPS”) 1008 is also used. SMPS 1008 is, for example, 12 Volts and 1.2 Amps. A switch 1010 controls whether or not SMPS 1008 is connected to the main control circuit. Main control circuit 1012 controls the information flowing in and out of body 1000. For example, main control circuit can be a microcontroller or a microchip. Main control circuit powers camera 1016 with 12 Volts of power via 1022. Camera 1016 records images of a living being's eye and sends the video signal 1014 back to main control circuit 1012. Main control circuit 1012 also controls when the stimulus system 1018 emits a stimulus via control signal 1020. For example, stimulus system 1018 is a light and the control signal 1020 is power to the light. A computer interface 1028 allows the body 1000 to interface with the computer. Video signals 1024 are sent from the camera 1016 via 1024 to the computer. The computer sends out a communications signal 1026 to the body 1000. This signal sends instructions to the camera body, such as to capture and record images of the eyes, start or stop the physical stimulus, change the intensity of the physical stimulus, change options on the cameras, and the like. For example, signal 1026 uses the RS-232C communications protocol.

In an embodiment of the present invention, the computer comprises an INTEL PENTIUM III™ (Intel Corporation) 400 MHz or faster processor is used. The computer further comprises 128 Mb or more of memory, 2 video capturing boards, and 2 cameras with a sensitivity of 0.05 lux. Moreover, the computer uses a WINDOWS 98/ME™ (Microsoft Corporation), WIN2000™ (Microsoft Corporation), or WINDOWS NT4 (Microsoft Corporation) operating system. The system uses the RS-232C communications protocol. The system is powered with 110/220 V at 50/60 Hz. The approximate weight of the camera system is 13 lb and the approximate size is 10×13×20 inches.

Dimensional changes of a living being's pupils reflect responses of the living being's autonomic nervous system caused by an external physical stimulus. A plot of the time-dependent dimensional changes (e.g., changes in the pupils radius) is known as pupillographic curve, pupil reflex curve, or simply, a pupillogram. The pupil reacts to physical stimuli, such as, light, sound, tactile contact, chemical irritant(s) or changes in temperature. Pupillary constriction and corresponding dilatation are involuntary reflexes under the control of the ANS.

An equation describing a pupillogram is: Dᵢ=Dₒ+ke^{αt}, where Dᵢ is initial pupil diameter, Dₒ is pupil width at the moment of maximal constriction, t is time, e is base of natural logarithm, and α and k are coefficients. Additional terms may be used to more accurately define the pupillogram. Inclusion of more parameters in the evaluation of a pupillogram may increase the effectiveness of the operation of the present invention. Accordingly, it is desirable to achieve a high level of diagnostic accuracy using analysis of a plurality of parameters characterizing a pupil's response to stimuli.

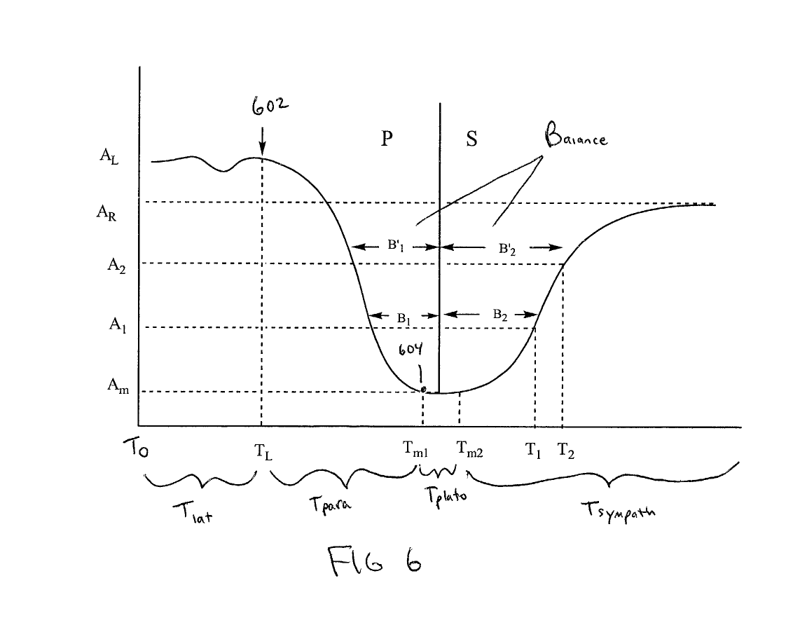

The coefficients that describe the mathematical model of a pupillogram like that shown in FIG. 6 may vary in number, for example between 4 and 10. These coefficients include latency time, time and speed of sympathetic and parasympathetic phases, dilation latency duration, recovery degree, and the like, which are further described herein.

In an embodiment of the present invention, dimensional changes of a living being's pupil, when stimulated once or on several occasions by a physical stimuli, are recorded. The living being's pupil may be photographed and a pupillogram of the pupil's response to the stimuli may be recorded and/or drawn. The pupillogram is created and then shown on the computer screen, such that the operator visually sees the pupillogram. Alternatively, the pupillogram is not drawn on the computer screen, but a hard copy of the pupillogram is printed out. Alternatively still, the pupillogram is not physically drawn for the operator to see, but is created internally, such as in the computer's memory. The computer uses the information it gathers and analyzes this information to evaluate the pupil of the living being. The computer may internally make a pupillogram, such as in its memory, without displaying the pupillogram, or may simply evaluate one or more parameters of the pupil.

Various parameters of the living being's pupil may be determined from the pupillogram, such as the initial pupil size, constriction latency time, parasympathetic phase amplitude, the parasympathetic phase speed (an average), parasympathetic phase gradient speed, the amplitude of minimal radius value, sympathetic phase latency period, sympathetic phase speed, the sympathetic period gradient speed and sympathetic phase amplitude. In one embodiment, these parameters are obtained from mathematical coefficients derived during the calculation of a pupillogram.

A living being's calculated coefficients (and thus characteristic parameters) are compared to normal living being's calculated coefficients (characteristic parameters) and ANS impairment is diagnosed by comparison. An embodiment of the present invention also provides a system that measures simultaneous dimensional changes of both pupils caused by an physical stimulus. Moreover, a pupillogram is provided that includes measurements of the living being's pupil, including measurements of the ANW. Additionally, dimensional changes at various locations of the living being's pupil, including the ANW or dimensional changes of an average of some specific parts of the living being's pupil, including the ANW, can also be measured.

FIG. 6 shows a pupillogram according to an embodiment of the present invention. In one embodiment of the invention, a pupillogram is calculated from dimensional changes based on the movement of a pupil border from any reference datum point ( usually center of iris or pupil, however, any reference point may be used to calculate the dimensional change of a pupil ). This can be done with a computer, wherein the exterior of the pupil is determined using edge detection software. The distance is electronically measured from the datum point to a plurality of points on the exterior of the pupil. In one embodiment, these distances are averaged over time to determine the dimensional changes of the pupil. In another embodiment, theses distances are summed together.

In addition, in one embodiment, the measurements of the pupil are normalized with the size of the iris of the eye. For example, the measurements of the pupil can be put in ratio with the measurements to the exterior of the iris. Thus, the distance the camera is from the eye is less relevant because of this normalization.

The measurements of the eye are continuously made over time. For each time period, the dimensional changes in the pupil are measured and calculated. In an embodiment, the computer analyzes the image of the pupil and calculates these measurements from the image. For example, the computer can find the plurality of points on the exterior of the pupil, and measure distances from those points to the datum point. The computer can repeat this analysis for each image of the pupil. Therefore, a pupillogram may obtained from dimensional changes averaged over the entire pupil or some parts thereof. Moreover, the system can also just look at portions of the pupil and look at dimensional changes in those portions of the pupil as opposed to the entire pupil.

In an embodiment, the system does not normalize or average dimensional changes in the pupil, but rather takes an actual measurement of the actual pupil's radius, diameter, circumference, or area. This can be accomplished by having the computer view the image of the pupil and record the actual size of the pupil. When measuring the actual size of the pupil, one or more photodiodes are used to calculate the distance from the camera to the eye, such that the actual measurement can be made. Alternatively, any other method of measuring the distance from the camera to the eye may be performed. The distance from the camera to the eye is useful for calculating the actual dimensional changes of the pupil.

A pupillogram represents a pupil histogram showing the average constriction and dilation of a pupil in response to a physical stimulus at time To. The vertical axis indicates the amplitude, radius, circumference, or area of the pupil, while the horizontal axis indicates time beginning at To or the time of the start of the physical stimulus.

A pupillogram may be divided into two portions labeled by “P” and “S” in FIG. 6. P is the parasympathic period and S is the sympathetic period. The parasympathetic period is the period in which the pupil is constricting in size due to the physical stimulus. The sympathetic period is the period in which the pupil is dilating or increasing in size, and thus recovering from the physical stimulus. The overall shape of the “curve” of the pupillogram corresponding to the “P” and “S” portions may be different, e.g. the pupillogram is not “symmetrical.” These deviations are quantified in the coefficients derived from the pupillogram and may be characteristic of each living being, a living being's ANS-impairment (caused for example by a drug), and the like.

Time Tₗₐₜ is the latency time of the pupil. This is the time it takes for the pupil to react to the physical stimulus by changing in size. Tₚₐᵣₐ is the parasympathetic time, which is the time the pupil is in the parasympathetic phase. The parasympathetic phase occurs when the pupil is constricting in size. Tₚₗₐₜₒ is the sympathetic phase latency time, which is the time during which the pupil is at its smallest size. T_{sympath} is the sympathetic time. The sympathetic time is the time during which the pupil is recovering from the physical stimulus and is increasing in size. A_{L} is the amplitude of the pupil during the latency period.

B_{alance} is a measurement of how fast the pupil constricts in size compared to how fast the pupil increases in size. The B_{alance} is measured by picking a certain amplitude, such as Aₗ. Aₘ is the amplitude of the smallest size of the pupil after being stimulated by the physical stimulus. The time it takes for the pupil to decrease in size from Aₗ to Aₘ is B₁. The time it takes for the pupil to increase in size from Aₘ to Aₗ is B₂. B_{alance} is the measurement of B₁/ B₂. By evaluating balance parameters, a determination may be made as to whether the living being is under the influence of drugs, has the proper aptitude for a specific position, whether the living being has suffered a brain trauma, or some other neuro-somatic disturbance.

FIG. 6 shows another pupillogram according to an embodiment of the present invention. The pupillogram represents a pupil histogram showing the average contraction and dilation of a pupil in response to a physical stimulus at time T₀. The vertical axis indicates the amplitude or radius of the pupil, while the horizontal axis indicates time beginning at T₀ or the time of the start of the physical stimulus. The amplitude or radius of the pupil may be an average of several measurements of the pupil, the distance between a fixed reference point (for example, the center) and one exterior point (such as the edge of the pupil), the distance between a fixed point a plurality of exterior points of the pupil, the circumference of the pupil, the area of the pupil, or the like. This fixed reference point may be inside the pupil, for example, at the center of the pupil.

Time T_{L} is the time at which the pupil begins contracting in response to the physical stimulus, at 602. T_{L}−T₀ is the pupil response latency time. A_{L} is the average radius of the pupil prior to the light flash. Tₘₗ is the time at which the pupil has contracted to the minimum radius, Aₘ. The time for the pupil to contract, excluding the latency time, is Tₘₗ−T_{L}, or Tₚₐᵣₐ. T₁ and T₂ are times chosen along the sympathetic portion of the pupillogram. Amplitudes A₁ and A₂ are the corresponding radii of the pupil at times T₁ and T₂.

Sₚ is the speed of the pupil contraction between T_{L} and Tₘₗ. D_{AB} is the linear distance along the curve of pupil contraction between T_{L} and Tₘₗ. % Aₙ, is the ratio of the pupil dilation distance at Tₙ (where n is an integer) in comparison to the total pupil contraction distance (A_{L}−Aₘ). Thus, %A₁ is the ratio of the pupil dilation distance at T₁ (which is A₁−Aₘ) over the average total pupil contraction distance (A_{L}−Aₘ). %A₂ is the ratio of pupil dilation distance at T₂ (A₂−Aₘ) to the average total pupil contraction distance (A_{L}−Aₘ). %Aₙ may change widely depending on the particular time point chosen which is of interest. In one embodiment of the present invention, time points corresponding to %Aₙ are in the range of 30-70%.

Information from the pupillogram can be used to determine whether a living being is under the influence of illicit drugs or alcohol. In addition, medicines containing substances that influence the autonomic nervous system, such as cholinomimetics, cholinolytics, adrenolytics, adrenoblockers, antihistamines, sedative, neuroleptics may also be detected. Moreover, based on collected data, the deviation a living being's parameters may be used to indicate exactly what substance the living being is intoxicated.

A living being that is under the influence of drugs will have a different pupillogram than the same living being when not under the influence of drugs. Alternatively, the pupillogram of the living being can be compared to a normal pupillogram, wherein the normal pupillogram is of normal living beings having similar characteristics to the living being, including, but not limited to physical characteristics such as age and sex. Additionally, the normal pupillogram can be taken under similar circumstances, such as distance of the camera to the pupil, amount of light, and the like.

A pupillogram of a living being under the influence of drugs has a slower than normal T_{para,} which is the time for the pupil to contract to the minimum radius (Tₘₗ). The pupillogram of a living being using alcohol is more flat in comparison to a normal living being, that is, D_{AB} is smaller for the alcohol user. Also, %A₁ and %A₂ are smaller in value for an alcohol user.

In evaluating a pupil according to an embodiment of the present invention, various parameters of the pupil are evaluated. In the present invention, one or multiple parameters in combination may be evaluated. These parameters, as shown for example in FIG. 6, include the following.

The initial pupil size: A_{L}. This parameter characterizes the size of the pupil prior to the pupil receiving a physical stimulus. The value for A_{L} can vary depending on the age of the living being that is being tested and physiological state of this living being. This parameter may be used to evaluate biological age of the living being, drug, such as alcoholic, intoxication of the living being, and cranio-cerebral disturbances of the living being.

Constriction latency time: T_{L}. This parameter characterizes the length of time the pupil takes to respond to the physical stimulus. For example, if using a light stimulus, this is the period from the moment the light flash contacts photo-receptors of eye retina, until the pupil starts constricting because of the light. This parameter may be used to evaluate the age of the living being, the depth and degree of nervous reaction of the living being, and drug intoxication of the living being.

Parasympathetic phase amplitude: A_{L}−Aₘ. This parameter characterizes the size difference between the initial and final pupil sizes. This parameter may be used to evaluate the depth and degree of primary reaction of the living being, the stability of the autonomic nervous system of the living being, and drug intoxication of the living being.

The parasympathetic phase speed: D/(Tₘₗ−T_{L}), where D is the distance between points 602 and 604. This parameter characterizes the speed that the size of the pupil takes to reach the parasympathetic maximum. This parameter is sensitive and thus varies depending on the presence of external influences on the autonomic nervous system, such as drugs.

Parasympathetic phase gradient speed: Sₚ. This parameter characterizes the coefficient that assists in the evaluation of the pupil for the presence and nature of discrete accelerations and decelerations on the pupil constriction region

The amplitude of minimal radius value: Aₘ. This parameter characterizes the size of the pupil when it is most constricted. This parameter allows the pupil to be evaluated to determine the degree of external influence, from example drugs. Further, this parameter may vary for living beings depending on their age.

Sympathetic phase latency period (the plateau): Tₘ₂−Tₘ₁. This parameter characterizes length of time the pupil remains at the size Aₘ. This parameter is sensitive and thus varies depending on the presence of external influences on the autonomic nervous system, such as drugs.

Sympathetic phase (the recovery period) speed: Ss. This parameter characterizes the average speed of pupil magnification. This parameter is useful because at different times, such as at Tₘ₂, Tₘ₁, T₁, and T₂, the sympathetic phase speed varies. Also this parameter varies between living beings based on their age and gender.

The sympathetic period gradient speed. This parameter characterizes the acceleration measured according to changes in the sympathetic phase speed. It may be measured using the pupillogram, such as by analyzing the pupillogram during the sympathetic period. The parameter may be used to evaluate whether the living being is under the influence of drugs.

Sympathetic phase amplitude. This parameter characterizes the increase in the pupil size after recovery from the constriction phase. The sympathetic phase amplitude at T₁ is shown as Aₗ−Aₘ and sympathetic phase amplitude at T₂ is shown as A₂−Aₘ.

The discrete recovery criteria at various time periods. This parameter measures the amount the pupil has recovered at various time periods, such as five time periods. For example, the pupil size can be measured at T₁ and T₂. Additionally, the pupil size can be measured at other times, such as T_{L}, TₘTₘ₂, or any other time period on the pupillogram.

The linear percent of pupil constriction. This parameter characterizes the percentage the pupil constricts due to the physical stimulus. It can be measured as a normalized value and shows the degree to which the pupil reacts during the parasympathetic phase.

Parasympathetic-sympathetic balance. This parameter is shown, for example, in FIG. 6. The parameter characterizes the degree to which the pupil has balance during the parasympathetic and sympathetic phases. It can be measured at a certain amplitude by the ratio of the time it takes for the pupil to decrease from the certain amplitude to Aₘ vs. the time it takes for the pupil to increase to the certain amplitude from Aₘ. The parasympathetic-sympathetic balance at A₁ is B₁/B₂. The parasympathetic-sympathetic balance at A₂ is B₁′/B2′. Alternatively, these ratios may be inverted such that at A₁ was measured as B₂/B₁ and the parasympathetic-sympathetic balance A₂ was measured as B₂′/B₁′.

The degree of recovery. This parameter characterizes the difference between the initial radius of the pupil and its radius at the end of the pupil reflex. For example, in FIG. 6 it is A_{R}/A_{L}. This parameter may be used to evaluate the reaction ability of the autonomic nervous system of the living being.

Angular parameters of the pupillographic curve. These parameters characterize the pupillographic curve at a specified angle. This parameter may be used to evaluate whether the living being is intoxicated by a drug. Additionally, this parameter may be effected by the age of the living being.

Simultaneous measurement of both pupils. This parameter reflects differences in the response of the left and right pupils of the living being. An embodiment of the present invention measures the reaction of either one or both pupils of the living being. When measuring both pupils, the embodiment can use binocular synchronous and symmetric measurements of both pupils and obtain the pupils'reactions in real-time.

The parameters described herein can be measured according to changes in the pupil in real-time. Thus, the video camera taking images of the pupils can feed this information into the computer such that the information is processed effectively immediately. The processed information can be displayed, used to create a pupillogram, analyzed, or the like, as the camera records the images. Therefore, there is little to no delay that occurs from the time the pupil reacts to the time the pupil's reaction is analyzed.

In another embodiment, the pupillogram, and the associated parameters can be displayed and calculated against time and converted into measurements against frequency using a Fourier transformation. In another embodiment, such parameters are measured against frequency and not against time. Using a pupillogram that is measured against frequency provides additional parameters that can be used to evaluate the living being and the pupil of the living being.

In operation of one embodiment, it is desired to detect the presence of a drug within a living being's body. The living being uses the viewfinder. While the living being is using the viewfinder, a camera is photographing and/or recording images of the pupils of the living being. The eye(s) of the living being are then subjected to a physical stimulus. From the time immediately prior to the physical stimulus being activated, the camera is recording images of the pupil(s) of the living being. These images are evaluated to determine parameters of the living being's pupil(s). This evaluation can occur as the pupil recovers from the stimulus or sometime after the pupil recovers from the stimulus. The evaluation can be of one of the pupils or both of (the left and right) the pupils. According to an embodiment of the present invention, pupillograms for a living being's left and right eyes can be taken in real-time, using a system of binocular synchronous and symmetric registration of both pupils' reaction. Binocular synchronous measurement of both eyes can occur simultaneously with the physical stimulus. Alternatively, a synchronous measurement of one of the eyes occurs simultaneously with the physical stimulus. Thus, the capturing of images of the eyes occurs at the same time as the physical stimulus is activated and/or deactivated.

To evaluate a pupil, the value of parameters D_{AB}, Sₚ, T₂−T₀, Tₘ, %A₁ and %A₂ for the pupil are obtained. One or more of these parameters are compared to a normal living being's values. The normal living being pupil's values are derived by evaluating a plurality of normal living being's pupil parameters for various living beings possessing various physical characteristics (such as age, gender, and the like), which is described herein. For example, the normal living being's pupil parameters can be an average of a plurality of normal living beings, such normal living beings numbering between 2 and 10,000, between 10 and 1,000, between 10 and 100, greater than 10, greater than 100, greater than 1,000, or the like. Statistical data regarding normal living beings can be collected on pupillogram parameters for various age groups, such as for groups spanning five year age intervals between the ages ranges of 5 to 81. Alternatively, the data can be grouped for every age or in ten year intervals. Thus, the living being could be within the average age of the normal living beings by within 5 years, 10 years, 1 year, or 15 years. Also, the data can be differentiated based on gender.

The characteristics of age and gender are good characteristics to use because pupillograms of living beings having similar age and gender tend to fall within the same ranges of error. Therefore, according to an embodiment of the present invention, the ranges of the parameters for normal living beings having similar age and gender are compared to the parameters for the living being. If the living being's parameters falls within the ranges of the normal living being parameters, then the living being is a normal living being. Otherwise, the living being is intoxicated with a drug or has some other condition affecting his or her ANS.

If the parameters of the living being differ from the parameters of the normal living being values, then drug use has been detected. In one embodiment, a margin of error may be used such the values may slightly differ and drug use will not deemed as having been detected. For example, the margin of error can be 1%, 2%, 5%, 10%, or 20%.

The determination of whether a parameter of a pupillogram of a living being is sufficiently different from a normal living being's pupillogram, can be set by the operator. The more a pupillogram of a living being deviates from the normal pupillogram, the more likely the living being is drug impaired. Moreover, a review of multiple pupillograms of living beings having known characteristics, allows the operator to determine the level of deviation that indicates drug impairment. For example, for a living being that is intoxicated with a drug, that living being's parasympathetic phase speed will be smaller than that of a normal living being. Additionally, for a living being that is intoxicated with a drug, that living being's latency time will be larger than that of normal living being.

In another embodiment, to determine whether a living being is under the influence of a drug, the sequential measurement and evaluation of the right and left pupils of the living being are determined and compared. A pupillogram of the right and left pupils of a living being under the influence of a drug may be different, while the pupillograms for the left and right pupils of a living being not under the influence of a drug are similar. Accordingly, the ratio of pupillogram curve parameters of the left and right pupil of a living being is a further method of determining actual or recent drug impairment by the living being. The ratio of curve parameters of a recent or presently drug impaired living being differs from the ratio of curve parameters of a normal living being.

Again, the parameters D_{AB}, S_{P}, T₂−T₀, Tₘ, %Al and %A₂ for the left and right pupil are computed using calculations described herein. The ratio of one or more of these parameters for the left and right pupil is then computed. If the ratios are larger or smaller than 1:1, within a margin of error, then drug use by the living being has been detected. In one embodiment the margin of error is 15% or less. In another embodiment, the margin of error is 20% or less. In another embodiment, the margin of error is 10% or less. In another embodiment, the margin of error is 5% or less. If the ratios are 1:1 within the margin of error, then drug use by the living being has not been detected.

Also, the comparison of the left and right pupil reaction to a physical stimulus can be measured using balance parameters. The parameters of the left and right pupil of a living being under the influence of drugs differs because the living being has less coordination between his or her left and right pupillary reactions. Accordingly, a difference between the parameters of the left and right pupil indicates drug use.

In another embodiment of the present invention, at least one of a living being's pupils is evaluated. The pupil can be evaluated as described above. Alternatively, both of the living being's pupils are evaluated. A period of time then elapses. For example, this period of time can be an amount of time greater than 30 seconds, greater than 1 minute, between 1 and 2 minutes, greater than 2 minutes, greater than 5 minutes, less than 10 minutes, less than 5 minutes, or any such time period. The at least one pupil of the living being is then evaluated again. The pupillogram from the first evaluation and the pupillogram from the evaluation that occurred after the time period are compared. When compared, a living being that is under the influence of drugs will have pupillograms that vary, while a normal living being will have pupillograms that are similar. While evaluating the pupils, any of the parameters of the pupil described herein can be used to evaluate the pupil. For example, during the comparison of the two evaluations (before and after the time period), any one or more of the parameters shown in FIG. 6 may be measured and compared.

Alternatively, after the living being's pupil are evaluated after the time period, a second time period may occur. The second time period may be the same duration of the first time period or may vary. After the second time period, a third evaluation of the pupil occurs. The results from the third evaluation are compared with the results of the first and/or second evaluation. Alternatively still, this process is repeated resulting in multiple evaluations, each separated by a time period. Alternatively still, the system varies which pupil is being evaluated. For example, first one pupil is evaluated, a time period elapses, and the other pupil is evaluated.

Alternatively, the system stimulates one pupil and evaluates the non-stimulated pupil. For a normal living being, the non-stimulated pupil will react similarly to the stimulated pupil. This allows for the simultaneous analysis of both direct and conjugate pupil reactions, where conjugate pupil reaction refers to dimensional changes and corresponding parameters of one pupil when the other is stimulated. For a living being affected by drugs, the conjugate pupil reactions will be different than conjugate pupil reactions for a normal person. This is because the presence of direct or crossed asymmetries is typical for living beings using drugs and living beings with disturbances of the central and peripheral nervous system.

When using a stimulus that is light, according to an embodiment of the present invention, the light may automatically provide one flash of light that is applied at a certain intensity and duration, in synchronous for both pupils, at the same time. Alternatively, the light can flash at various intervals, either in synchronous or out of synchronous for each pupil. Alternatively still, the intensity and duration can change throughout the evaluation.

When evaluating the pupil of a living being, the evaluation can be used to determine whether the living being is under the influence of a drug, whether the living being possesses certain aptitudes, or the like. When evaluating the aptitude of a living being, such as for vocational skills, the parameters of the pupil are compared with the parameters of a pupil of a normal living being of similar physical characteristics. The parameters of the living being's pupil can be evaluated to determine if it is faster or quicker than the normal living being. An operator can determine which parameters are important for a particular aptitude and then evaluate the pupil of the living being to decide whether the living being possesses the qualities that are desired. For example, it may be desired for a living being that is to be in the public service to have a faster than normal response time to adjust to significant changes in the amount of light. The embodiment of the present invention can be used to evaluate the pupils of the living being to make this determination.

Prior to having its pupil(s) evaluated, the living being can register with the system of the present invention. During registration, the living being may provide the system with his or her age, gender, height, weight, and other physical characteristics. This information may be used to determine the normal response for this living being. Additionally, the system can store information on the living being's pupils and use this information for future evaluations of the living being's pupil(s). For example, after registering and having its pupils evaluated, the system may determine that the living being's pupils were normal. On a subsequent evaluation, the system may compare the results of the normal results, or alternatively, with a subsequent evaluation of the same living being. If the comparison between the subsequent and prior evaluations differed, then the system could determine that the living being was now under the influence of drugs.

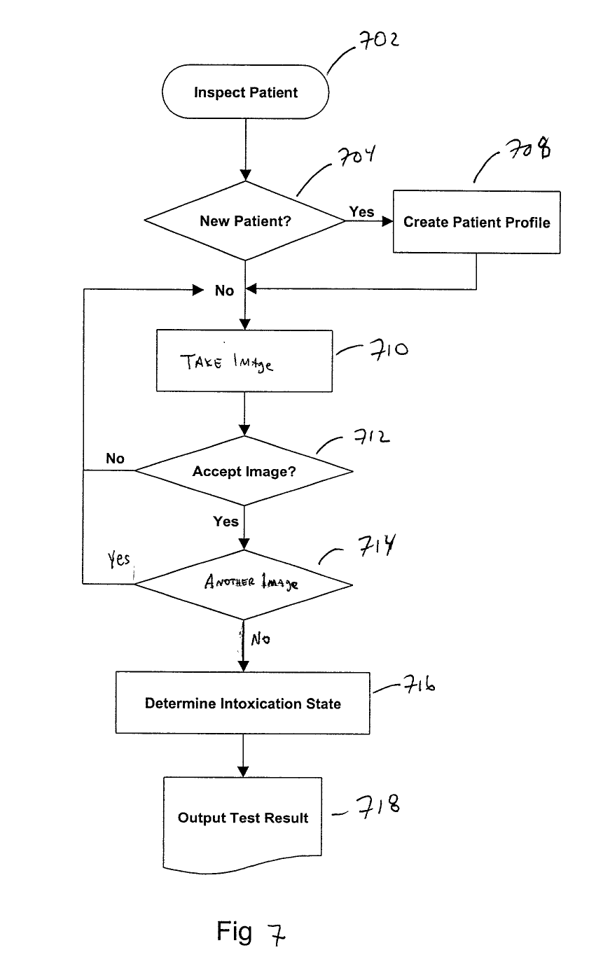

FIG. 7 shows a flow chart of an embodiment of the present invention. At 702 a living being is selected, volunteers, or is chosen to be inspected by the embodiment of the present invention. The operator or system inquires whether this living being is a new patient at 704, e.g., never used the system before. If it is a new patient, at 708, the living being registers with the system as described herein by entering information regarding the living being, such as his or her age, gender, eye color, weight, height, and the like. This information is used to in a profile of the patient.

At 710 a physical stimulus is emitted by the system on the eyes of the living being. Cameras in the system record the reaction of the pupil and/or ANW of the living being. The system can capture images of one or both eyes of the living being, as selected by the operator. Alternatively, as discussed herein, the system can stimulate one eye and record images of the other eye.

At 712, the operator has an opportunity to view the images of the living being's eye(s) recorded by the system. This can occur in real-time, such that the operator sees the images on the computer as the camera records the images, or may be delayed. The operator may also see pupillograms of the pupils or ANW of the living beings eyes on the computer.

At 712, the operator has the option to accept or reject the image. If the operator rejects the image, the system repeats the process at 710. If the image is acceptable, the system asks if another image is to be taken at 714.

At 714, the operator may select to have another image taken of one or both of the eyes of the living being. If desired to take another image, the system can wait a predetermined amount of time, as described herein, or may take the image immediately. If another image is to be taken, the system returns to steps at 710. If another image is not to be taken, the system progresses to step 716. The system can take as many images as desired by the operator by repeating steps 710, 712 and 714.

At 716, the system automatically evaluates the living being. Alternatively, the evaluation of the living being is done manually by the operator. The system can analyze the pupillogram(s) taken of the living being and compare then with normal pupillogram(s) from normal living beings having similar characteristics to the living being or may compare the pupillogram(s) with each other, as described herein. Such evaluation can determine if the living being is intoxicated with a drug. Alternatively, the evaluation can determine whether the living being is suitable for a certain task.

At 718, the results are output. The output may be in the form of a printout, may be viewed on the screen, may be electronically stored in the computer, or the like. The output can contain the pupillograms that were created during the evaluation, particular parameters of the pupillogram that the system evaluated, the results of the evaluation, and the like.

In another embodiment, the camera system is setup such that two video cameras are used, one for each pupil of the living being. Each camera is setup at the same distance from the pupil, and each pupil receives the same amount of light. Additionally, there may be multiple frame grabbers, one for each camera, to grab images from the camera. These two cameras can also be synchronized. Alternatively, one camera is used to photograph one or both pupils. Further, images from the camera(s) may be transmitted and displayed on the computer in real-time. Moreover, the camera system can additionally include a device for providing the physical stimulus, such as a light, illuminator, or the like. Further, the camera system can also include a video blaster or frame grabber.

The camera system can be setup with two cameras such that the length of the channels from each camera to the lens is the same. When the camera is adjusted, the channels can remain the same for each camera. Therefore, the systems linear and adjustment parameters can also be identical.

In the channels of the camera system, the resolution depth of each channel can be greater than or equal to 12 mm. This allows room for the curvature of the living being's eyes. Alternatively, the resolution depth of each channel can be any size that is desired by the operator.

In another embodiment, a separate stimulator, such as an illuminator, can be installed in each optical channel. Therefore, each illuminator stimulating one pupil. Alternatively, one illuminator may be used to stimulate both pupils.

In another embodiment, IR diodes are used. For example, the IR diode can be a multiple wavelength IR diode or an infrared emitting diode (“IRED”), such as shown in U.S. Pat. No. 5,187,506, which is herein incorporated by reference in its entirety. The IR diodes can be used in conjunction with the video camera. This provides the benefits of both IR diodes and a video camera to be obtained. The IR diodes allow for images to be recorded of the pupil, without the iris pigment saturation and its reflecting properties effecting the images. For example, irises that differ in color emit wavelengths of light that vary in length. The diodes may be used to automatically adjust the system for any deviations that occur due to the eye color of the living being. Additionally, the system may detect the color of the iris and then use software to adjust for variances of measurements caused by the colors of the iris. Alternatively, the system can be used to detect the exact eye color of the living being, down to the nanometer.

In another embodiment, the system can adjust the physical stimulus to account for the eye color of the living being. The system or operator can ask the living being prior to beginning what their eye color is, or the system can detect the eye color of the living being as described herein. For example, the standard physical stimulus (that is for example a light) may use 880 nm and 940 nm LEDs. However, the wavelength of the light emitted may be adjusted according to color of the iris of the living being to generate the best image. Additionally, adjusting the wavelength of the light also prevents refraction of light within the iris.

In another embodiment, the physical stimulus can be adjusted such that it is diffused. For example, with a stimulus that is illumination, diffused illumination may be used to prevent reflection within the eye. This would prevent any variances of measurements that were caused by refraction or reflection of light within the eye.

In one embodiment, the measured parameters of the pupillogram are stored in the computer, such as in the memory of the computer. Additionally, an evaluation of the pupillogram can also be stored. Newly processed data regarding newly derived pupillograms can be compared to stored data regarding pupillograms.

In another embodiment, the system can automatically adjust the camera and or the viewfinder to obtain an image wherein the pupil is centered. This can also be done via software, such that after the image is recorded, the software automatically centers the image. The system can alternatively center the image based on the eye, instead of just the pupil. The system centers the eye and/or pupil by positioning the eye and/or pupil perpendicular to the axis of the optical channel.

In another embodiment, the computer can automatically control the physical stimulus such as to start at a certain time, to occur synchronously for each pupil, or to occur at a different time than the other pupil's stimulus. Alternatively, one stimulus is used on both pupils. Further, the stimulus can be setup to automatically start in synchronous with the camera's synchronization. The system can thus be synchronized with the camera and stimulus in each channel separately or via both channels. Alternatively, the operator can control the stimulus and/or the camera starts.

Alternatively, the intensity and duration of the physical stimulus can be adjusted according to the living being, such as based on the age and gender of the living being. The stimulus may have a default value for its intensity and duration, and these defaults values can be adjusted automatically by the computer based on the physical characteristics of the living being or manually by the operator.

In another embodiment, both photodiodes and video cameras are used for evaluating the pupils. Using both photodiodes and video cameras allows for multiple measurements of the parameters of each pupil.

Additionally, two video frame grabbers are used for sending information regarding the pupils to the computer. This allows for the input and analysis of both pupils simultaneously in real-time. Alternatively, one video frame grabber may be used such that it switches between grabbing a frame for one pupil and then grabbing a frame for the other pupil. When using one video frame grabber, it may be more efficient for the frame grabber to alternative quickly between the pupils.

In another embodiment of the invention, dimensional changes corresponding to changes in pupil radius are divided by corresponding dimensional changes in the iris ratio. This normalized method for pupillogram calculation has the advantage even if image sizes of the images evaluated change, such changes do not themselves lead to changes in the pupillogram.

Using an embodiment of the present invention, measurement of pupil images occur such that images are recorded at a rate of, for example 25-30 frames per second.

The present invention will be described in more detail by way of the following examples. These examples are provided for only illustration and should not be construed as limiting the scope of the invention, which is properly delineated in the accompanying claims.

Test were run on living beings that were addicted to drugs and on living beings that were otherwise healthy. The results of the test appear in Table 1 herein.

TABLE 1

| | | | | AVERAGE |
|---|---|---|---|---|
| | DRUG | HEALTHY | HEALTHY | POPU- |
| GROUP | ADDICTS | PERSONS | PERSONS | LATION |
| AGE | 14-35 | 9-15 | 16-25 | 25+ |
| | Yrs. Old | Yrs. Old | Yrs. Old | Yrs. Old |
| NO. TESTED | 473 | 172 | 675 | 331 |
| NORMAL | 19.1% | 84.9% | 75.9% | 71% |
| UNDEFINED | 12.5% | 11.4% | 16.7% | 26% |
| INTOXICATED | 68.4% | 3.7% | 7.4% | 3% |

As shown in Table 1, 473 living beings, aged 14-35 years of age were tested according to an embodiment of the present invention. Of these living beings, 68.4% of them tested positive for drugs. The living beings that tested positive for drugs were evaluated by a trained physician to determine that 98% of these living beings were actually correctly identified as drug users. The other 2% were identified as “undefined.” Being characterized as undefined included many symptoms, such as a brain trauma (either as acquired at birth or subsequently acquired), diseases that bear symptoms closely related to that of drug use, brain damage from toxins, or the like.

Further, of the 473 living beings that were admitted drug addicts, under the embodiment of the present invention, 12.5% of these living beings were identified as undefined. Upon being examined by a trained physician, these individuals were identified as follows: 67% of them were diagnosed as drug addicts, 28% were characterized as undefined, and 5% of them were characterized as being normal. Being characterized as being normal means that the living being is not a drug user.

Of the 473 living beings that were admitted drug addicts, under the embodiment of the present invention, 19.1% of these living being were identified as normal. Upon being examined by a trained physician, 95% of these individuals were diagnosed as normal, with the other 5% being diagnosed as undefined.

172 living beings between the ages 9-15 years of age were admittedly declared as being non-drug users, or otherwise healthy, and were tested using an embodiment of the present invention. Of these people, 84.9% were tested as being normal, 11.4% were tested as being undefined, and 3.7% were tested as being intoxicated. 675 living beings between the ages of 16-25 years of age were admittedly declared as being non-drug users, or otherwise healthy, and were tested using an embodiment of the present invention. Of these people, 75.9% were tested as being normal, 16.7% were tested as being undefined, and 7.4% were tested as being intoxicated.

Further, 331 living beings over the age of 25 were of the average populate, randomly selected, were tested using an embodiment of the present invention. Of these people, 71% were tested as being normal, 26% were tested as being undefined, and 3% were tested as being intoxicated.

When serious drug addicts were diagnosed using a blood test, urinalysis, or other such conventional drug screening tests, and if such drug addicts have not taken drugs for over 3 days, these drugs addicts are almost all diagnosed as not being intoxicated. Using an embodiment of the present invention, 479 drug addicts were diagnosed, with some of these drug addicts having abstained from drugs for a period of time between 1 week and 2 months. After testing according to an embodiment of the present invention, 76% of these drug addicts were diagnosed as having been intoxicated with drugs. Accordingly, an embodiment of the present invention has increased reliability in detecting drug use by a living being, even after a number of days have elapsed. The number of days can be, for example, any number of days up to 2 months, up to 1 month, or up to 1 week.

According to another example of the present invention, pupils of various athletes were analyzed according to the present invention to determine whether they were suited for particular sporting positions. The results were confirmed using a thorough medical examination of the individuals.

In this example, 60 male athletes of 17-18 years of age in the following sporting specializations were examined: 37 athletes from the stamina group (skiing, swimming, and rowing); 12 athletes from the game sports group (volleyball, basketball, football, and hockey); and 11 athletes from the speed-power sports group (wrestling and boxing). All of these athletes were entrants in the Moscow Sports Academy and had passed standard clinical and laboratory examinations, had a medical examination confirming that they were fit to perform in their respective professional sports and had no known health problems.

Each athletes' pupils were analyzed in response to a stimulus according to an embodiment of the present invention. During each examination, three series of pupillary responses to a standard light stimulus were made. The duration of each examination was 2.5 seconds and the interval between measurements was about 60 seconds. The duration of the light stimulus was 30 ms. Physical loading was conducted according to generally accepted 9-minute European veloergometer test.

The following parameters were measured in addition to pupillometry examination before physical loading and at 6 to 8 minutes after loading:

1. Anthropometrics (height (cm), body mass (kg));

2. Respiratory capacity (ml³) (“RC”);

3. Hypoxic test (sec) (breath holding at inhalation (“BHI”));

4. Cardiovascular system:

a. Heart rate (1/min) (“HR”);

b. Arterial pressure (mm of mercury) (“AP”);

c. Parameters of electrocardiography;

5. Nervous system:

a. Reaction rate—chronoreflexometry (msec) (“CRM”);

b. Tremorometry (err/min) (“tremor”);

c. Muscular contraction rate (1/min) (“MCR”);

6. Reflex diagnostics-average parameter of electric potential (“EP”);

7. The parameter of health level—Machalanobis index (“MI”); and

8. PWC working capability (absolute—watt (W) and relative—Watt/Kg (W/Kg) parameters of working capability).

All the parameters were statistically processed and were represented as M+in.

The athletes were grouped on the basis of their results. The first group included 46 athletes (76.6 %) who had steady pupillometric parameters before and after the physical loading. All parameters (starting capabilities, amplitude of response on light stimulus, sympathetic-parasympathetic balance, degree of recovery after light stimulus, velocity vector) complied with the athletes' age norm.

TABLE 2
Parameters of pupillary reaction state before loading

| | Name of | | | Re- | | | |
|---|---|---|---|---|---|---|---|
| | sports | Number | | sponse | Velo- | Re- | Re- |
| | special- | of sub- | Starting | ampli- | city | covery | covery |
| | izations | jects | index | tude | vector | degree | index |
| 1 | Game | 25 | 85 ± | 66 ± | 85 ± | 75 ± | 75 ± |
| | | | 1.3 | 3.2 | 0.4 | 2.8 | 0.3 |
| 2 | Stamina | 12 | 85 ± | 52 ± | 75 ± | 62 ± | 75 ± |
| | | | 1.8 | 1.6 | 1.1 | 1.7 | 0.7 |
| 3 | Speed- | 9 | 85 ± | 66 ± | 75 ± | 50 ± | 55 ± |
| | power | | 1.2 | 2.5 | 0.9 | 2.1 | 0.6 |

According to the data of Table 2, starting capabilities of the vegetative nervous system of the athletes were high enough and stable enough for athletes of all the groups. In this example, the following parameters were measures: the starting index (which is the initial pupil condition); the response amplitude (which is the constriction of the pupil); the velocity vector (which was the parasympathetic and sympathetic velocity); the recovery degree (which was the recovery of the pupil size after a period of time); and the recovery index (which is an index of how the pupil responded to the stimulus, including information relating to the recovery degree, recovery speed, and gradient speed). The amplitude of response from the light stimulus was lower for the stamina group as opposed to the other groups, this is because of the need of athletes in this group to save strength during long periods. The velocity vector was higher for members of game sports group, reflecting their need for fast and nonstandard decision-making. The recovery degree and index was highest for the game and stamina groups. These parameters were lower for the athletes of the speed-power group, because these sports require more muscle-nervous response for short periods of time with relatively slow recovery.

TABLE 3
Parameters of cardio-respiratory and nervous systems before loading

| Name | | | | | | | | | | | | |
|---|---|---|---|---|---|---|---|---|---|---|---|---|
| of | | | | | | | | | | | | |
| sports | | | | | | | | | | Ap min, | AP max, | |
| speciali- | Height, | Mass, | RC, | BHI, | CRM, | Tremor, | | | HR, | mm of | mm of | MCR, |
| zation | cm | kg | mm³ | sec | msec | err/min | EP | MI | 1/min | mercury | mercury | 1/min |
| Stamina | 174.5 ± | 66.3 ± | 5.1 ± | 66.4 ± | 0.127 ± | 0.83 ± 0.05 | 92 ± 3.8 | 1.1 ± 0.09 | 70.8 ± 3.1 | 60 ± 3.1 | 100 ± | 97.0 ± 2.75 |
| | 2.1 | 3.2 | 1.4 | 2.1 | 0.003 | | | | | | 4.8 | |
| Game | 183.8 ± | 75.6 ± | 4.75 ± | 63.2 ± | 0.194 ± | 0.92 ± 0.09 | 83.3 ± 3.1 | 1.12 ± 0.064 | 69.8 ± 5.9 | 72 6 ± 4.1 | 121.6 ± | 112 2 ± 4.8 |
| | 4.2 | 6.1 | 1.2 | 1.5 | 0.009 | | | | | | 4.8 | |
| Speed- | 172.3 ± | 71.3 ± | 4.4 ± | 70.1 ± | 0.081 ± | 1.19 ± 0.09 | 78.1 ± 2.89 | 1.02 ± 0.07 | 67.4 ± 4.7 | 71.5 ± 3.4 | 120 ± | 954 ± 9.7 |
| power | 3.1 | 4.6 | 1.2 | 3.9 | 0.011 | | | | | | 4.6 | |

The parameters in Table 3 show that cardio-respiratory system state including ECG data were within normal limits for the athletes. The parameters of nervous system response, taking into account the type of sports, were higher in the game sports group than in speed-power sports and stamina groups, which corresponds to higher adaptive reaction and is confirmed by a higher MCR.

TABLE 4
Parameters of pupillary reaction state after loading

| | Name of | | | Re- | | | |
|---|---|---|---|---|---|---|---|
| | sports | Number | | sponse | Velo- | Re- | Re- |
| | special- | of sub- | Starting | ampli- | city | covery | covery |
| | izations | jects | index | tude | vector | degree | index |
| 1 | Game | 25 | 82 ± | 65 ± | 84 ± | 75 ± | 74 ± |
| | | | 2.5 | 2.8 | 0.7 | 1.3 | 0.8 |
| 2 | Stamina | 12 | 86 ± | 51 ± | 75 ± | 62 ± | 74 ± |
| | | | 2.1 | 2.1 | 2.2 | 2.0 | 1.3 |
| 3 | Speed- | 9 | 83 ± | 64 ± | 75 ± | 50 ± | 54 ± |
| | power | | 2.2 | 1.8 | 1.6 | 1.6 | 1.2 |

The parameters of pupillary reaction before and after physical loading fluctuated within the limits of average arithmetic error, indicating the stability of vegetative regulation.

TABLE 5
Examined parameters after physical loading

| Name | | | | | | | | | | | | |
|---|---|---|---|---|---|---|---|---|---|---|---|---|
| of | | | | | | | | | | | | |
| sports | | | | | | | AP max, | AP min, | | | | |
| speciali- | RC, | BHI, | MCR, | CRM, | Tremor, | HR, | mm of | mm of | | | PWC, | PWC, |
| zations | mm³ | sec | 1/min | msec | err/min | 1/min | mercury | mercury | EP | MI, max | W | W/Kg |
| Stamina | 4.8 ± | 70.3 ± | 105.1 ± | 0 098 ± | 0.88 ± | 9 24 ± | 130 ± 3.65 | 65 ± 3.1 | 138 ± 5.03 | 1.0 ± 0.03 | 181.2 ± 4.1 | 2 4 ± 0.1 |
| | 1.3 | 4.1 | 3.1 | 0.011 | 0.07 | 1.2 | | | | | | |
| Game | 4.9 ± | 63.3 ± | 112.4 ± | 0.104 ± | 0.9 ± | 127 ± | 136.4 ± 3.4 | 82.7 ± 2.3 | 136 8 ± 3.4 | 0.81 ± 0.03 | 147.4 ± 4.1 | 1.99 ± 0.09 |
| | 0.77 | 3.9 | 1.8 | 0011 | 0.11 | 2.3 | | | | | | |
| Speed- | 4.28 ± | 66.4 ± | 100.4 ± | 0.086 ± | 1.02 ± | 85.1 ± | 120.5 ± 3.8 | 68 ± 2.1 | 121.3 ± 3.8 | 0.75 ± 0.03 | 148.9 ± 6.1 | 2.2 ± 0.09 |
| power | 1.2 | 3.9 | 6.1 | 0.009 | 0.01 | 3.4 | | | | | | |

Tables 3 and 5 show, that the parameters were adequate for physical loading. Recovery was sufficient, without signs of fatigue. Almost all the athletes had average parameters of working capability, about 2 W/Kg, that correspond to 12 kg/m/kg. All athletes recovered after 10 minutes.

According to Nacatany's reflex diagnostics, the average value of electric potential on meridians of 120 microvolts showed that adaptation to the loading was correct and the loading did not cause inadequate changes in the organs or systems. The Machalanobis health index before and after the loading had not significantly varied, which indicates the absence of pre-pathologic changes in organs and systems, which can be detected even if there were no changes in the resting condition.

The second group included 14 living beings (23.3%). The changes of pupillary unconditional reflex were detected in the athletes of this group by pupillometry. Three subgroups (2a, 2b, 2c) were formed according to the degree of pronunciation of pupillographic parameters changes.

Seven (11.6%) athletes were included in subgroup 2a. Their pupillograms had normal characteristics before physical loading, but after the veloergometer test a decrease of the recovery process in the sympathetic phase was detected, including a decrease in the response amplitude and start index, which indicated a decrease in the nervous system reserve.

TABLE 6
Parameters of athletes with decreased nervous system reserves

| | | | | | | | AP, | | | | |
|---|---|---|---|---|---|---|---|---|---|---|---|
| | RC, | BHI, | MCR, | CRM, | Tremor, | HR, | mm of | | | PWC, | PWC, |
| Parameters | mm³ | sec | 1/min | msec | err/min | 1/min | mercury | EP | MI | W | W/Kg |
| Before Phys. | 4.3 ± 0 9 | 4 1 ± 2.4 | 72 ± 2.8 | 0.075 ± | 0.65 ± 0.01 | 65 ± 1.4 | 120 ± 1.4 | 76.5 ± 1 3 | 1.1 ± 0.2 | 1 | |
| loading | | | | 0.008 | | | | | | | |
| After Phys | 4.0 ± 0.9 | 35.1 ± 2.1 | 68 ± 3.1 | 0.203 ± | 1.40 ± 1.2 | 140 ± 3.1 | 140 ± 1.9 90 | 80.1 ± 2.1 | 3.1 ± 0.35 | 176 ± 3.8 | 2.32 ± 0.5 |
| loading | | | | 0.01 | | | ±1.7 | | | | |

The parameters in Table 6 show that initial parameters in this subgroup had no deviations from the normal average except for a slightly increased Machalobis index. However, after physical loading the following was shown:

An increase of the Machalobis index;

Relatively low electric parameters;

A decrease of MCR;

Slowing down of the reaction (CRM); and

An increase in the tremorometry parameters.

The disturbances of cardiovascular system and pancreas were detected in this group by the “Diacoms” method. Reflex-diagnostics was detected in one athlete showing a considerable CNS over-tension rather than a depression of ECG wave TV5-V6.

Subgroup 2b was formed with 4 athletes (6.7%), who, according to pupillometry data, had moderate instability of pupillary reactions, a considerable decrease of nervous system reserves, low starting capabilities, and poor parameters recovery both base (before loading) and after loading.

The data of other examination methods (see Table 7) and “Diacoms” reflex-diagnostics had confirmed that these athletes were in the over-training state with pronounced vegeto-vascular changes. Pronounced arrhythmia is manifested in ECG, wave TV4-V6 was smoothed. Relative working capability in this subgroup was low.

TABLE 7
Parameters before and after loading

| | | | | | | | AP max, | AP min, | | | | |
|---|---|---|---|---|---|---|---|---|---|---|---|---|
| Para- | RC, | BHI, | MCR, | CRM, | Tremor | HR, | mm of | mm of | | | PWC, | PWC, |
| meters | mm³ | sec | 1/min | msec | err/min | 1/min | mercury | mercury | EP | MI | W | W/Kg |
| Before | 3.3 ± 0.8 | 22 ± 2.9 | 40 ± 2.1 | 0.325 ± 0.009 | 1.6 ± 0.5 | 60 ± 1.8 | 100 ± 2.9 | 60 ± 3.75 | 55 ± 4.1 | 3.8 | 70 | 1.3 |
| Phys. | | | | | | | | | | | | |
| Loading | | | | | | | | | | | | |
| After Phys | 3.0 ± 0.5 | 20 ± 1.8 | 30 ± 3.1 | 0.500 ± 0.01 | 2.8 ± 0.4 | 160 ± 3.5 | 170 ± 4.1 | 90 ± 1.9 | 61 ± 3.3 | 4.1 | — | 0.04 |
| loading | | | | | | | | | | | | |

High AP and HR after 7 to 8 minutes of physical loading showed intensive recovery processes in these athletes. Further observation of this subgroup has shown that ECG and AP parameters came to be normal after one day.

Three people (4.7%) were included into subgroup 2c. They showed pronounced instability of pupillographic parameters both before and after physical loading, showing a sharp decrease of the starting index, response amplitude, velocity vector, degree and index of recovery specifying disturbances of vegetative nervous regulation. These athletes showed signs of the effect of exogenous toxic substances.

These results show a high degree of correlation between the pupillometry data obtained using an embodiment of the present invention with the data of a complex medical examination that was used to estimate the athletes' functional state. Pupillographic parameters are thus informative of an estimation of the nervous system resources, the state of vegetative regulation, the adaptive capabilities, recovery rate and training degree for living beings. Therefore, an embodiment of the present invention can be used to monitor a living being in the practice of sports medicine. Additionally, an embodiment of the present invention can be used to:

Detect hidden pathology in CNS, VNS, psychosomatic disturbances;

Evaluate the proper sport for a living being to compete;

Detect a proper training level for a living being; and

Adjust the training process of living beings during different preparation stages.

The methods and apparatus of the present invention, or certain aspects or portions thereof, may take the form of program code (e.g., instructions) embodied in tangible media, such as floppy diskettes, CD-ROMS, hard drives, or any other machine-readable storage medium, wherein, when the program code is loaded into and executed by a machine, such as a computer, the machine becomes an apparatus for practicing the invention. The methods and apparatus of the present invention may also be embodied in the form of program code that is transmitted over some transmission medium, such as over electrical wiring or cabling, through fiber optics, or via any other form of transmission (such as an electronic connection), wherein, when the program code is received and loaded into and executed by a machine, such as a computer, the machine becomes an apparatus for practicing the invention. When implemented on a general-purpose processor, the program code combines with the processor to provide a unique apparatus that operates analogously to specific logic circuits.

The above steps depicted in flow charts and methods herein may be performed in a different order than as depicted. The steps shown are merely exemplary of the order these steps may occur. The steps shown herein may occur in any order that is desired, such that it still performs the goals of the claimed invention. Additionally, steps not desired to be used from the steps shown in the flow charts and methods may be eliminated, such that the goals of the claimed invention are still achieved.

All patents and publications described herein are hereby incorporated by reference to the same extent as if each individual patent or publication was specifically and individually indicated to be incorporated by reference.

The inventions illustratively described herein can suitably be practiced in the absence of any element or elements, limitation or limitations, not specifically disclosed herein. Thus, for example, the terms “comprising,” “including”, “containing”, etc., shall be read expansively and without limitation. Additionally, the terms and expressions employed herein have been used as terms of description and not of limitation, and there is no intention in the use of such terms and expressions of excluding any equivalents of the future shown and described or portion thereof, but it is recognized that various modifications are possible within the scope of the invention claimed. Thus, it should be understood that although the present invention has been specifically disclosed by preferred embodiments and optional features, modification and variation of the inventions embodied therein can be used by those skilled in the art, and that such modifications and variations are considered to be within the scope of the inventions disclosed herein. The inventions have been described broadly and generically herein. Each of the narrower species and subgeneric groupings falling within the generic disclosure also form part of these inventions. This includes the generic description of each invention with a proviso or negative limitation removing any living being matter from the genus, regardless of whether or not the excised materials specifically resided herein. Other embodiments are within the following claims. In addition, where features or aspects of an invention are described in terms of the Markush group, those schooled in the art will recognize that the invention is also thereby described in terms of any individual member or subgroup of members of the Markush group.

From the description of the invention herein, it is manifest that various equivalents can be used to implement the concepts of the present invention without departing from its scope. Moreover, while the invention has been described with specific reference to certain embodiments, a person of ordinary skills in the art would recognize that changes can be made in form and detail without departing from the spirit and the scope of the invention. The described embodiments are to be considered in all respects as illustrative and not restrictive. It should also be understood that the invention is not limited to the particular embodiments described herein, but is capable of many equivalents, rearrangements, modifications, and substitutions without departing from the scope of the invention.

What is claimed is:

1. A method of evaluating a living being, said method comprising the steps of:
stimulating an eye of a living being using one or more physical stimulus, wherein said eye comprises a pupil;
electronically measuring a dimensional change in said pupil in response to said at least one physical stimulus;
automatically determining at least one parameter relating to said dimensional change;
electronically storing said at least one parameter in a computer;
determining at least one normal parameter, wherein said normal parameter comprises information relating to a plurality of other normal living beings that have at least one similar characteristic of said living being, wherein said at least one similar characteristic is age or gender;
comparing said at least one parameter with said at least one normal parameter; and
evaluating said living being based on said comparison step.

2. The method of claim 1, further comprising automatically creating a pupillogram based on said dimensional change in said pupil in response to said physical stimulus.

3. The method of claim 2, wherein said step of automatically creating said pupillogram comprises:
recording said dimensional change in said pupil using a video camera; and
automatically creating said pupillogram in real time.

4. The method of claim 3, wherein said pupillogram is based on frequency.

5. The method of claim 3, further comprising:
displaying an image of said pupil on said computer in real time; and
displaying said pupillogram on said computer in real time.

6. The method of claim 1, wherein said step of measuring comprises synchronous measurement of both of said pupils of said living being with said step of stimulating.

7. The method of claim 1, wherein said step of evaluating said living being comprises:
determining whether said living being is impaired by at least one drug.

8. The method of claim 1, wherein said step of evaluating said living being comprises:
determining whether said living being is suited for a particular vocational or athletic task.

9. The method of claim 1, wherein said step of electronically measuring comprises:
electronically measuring a first distance comprising the distance from a datum point to a point on the exterior of said pupil;
electronically measuring a second distance comprising the distance from a datum point to a second point on the exterior of said pupil; and
automatically averaging said first distance and said second distance.

10. The method of claim 1, wherein said parameter comprises one or more of the following: initial pupil size, constriction latency time, parasympathetic phase amplitude, parasympathetic phase speed, parasympathetic phase gradient speed, amplitude of minimal radius value, sympathetic phase latency period, sympathetic phase speed, sympathetic period gradient speed, and sympathetic phase amplitude.

11. The method of claim 10, wherein said parameter comprises at least three of the following: initial pupil size, constriction latency time, parasympathetic phase amplitude, parasympathetic phase speed, parasympathetic phase gradient speed, amplitude of minimal radius value, sympathetic phase latency period, sympathetic phase speed, sympathetic period gradient speed, and sympathetic phase amplitude.

12. The method of claim 1, wherein the physical stimulus comprises one or more of the following: light, sound, tactile contact, chemical irritant and temperature change.

13. The method of claim 1, further comprising:
stimulating a second eye of said living being using one or more physical stimulus, wherein said second eye comprises a second pupil;
electronically measuring a dimensional change in said second pupil in response to said at least one physical stimulus at the same time as said dimensional change in said first pupil is electronically measured;
automatically determining at least one parameter relating to said dimensional change of said second pupil;
comparing said at least on parameter of said second pupil with said at least one normal parameter; and
automatically evaluating a autonomic nervous system of said living being based on said comparison steps.

14. The method of claim 1, wherein said physical stimulus comprises the removal of one or more of the following: light, sound, physical contact, chemical irritant or temperature irritant.

15. The method of claim 1, wherein said age of said living being and an average age of said other normal living beings are within 10 years of each other.

16. The method of claim 1, wherein said age of said living being and an average age of said other normal living beings are within 5 years of each other.

17. The method of claim 1, wherein said physical stimulus is light, and said light is adjusted according to an eye color of said living being.

18. A method of evaluating a living being, said method comprising the steps of:
stimulating at least one eye of a living being using a first physical stimulus, wherein said at least one eye of said living being comprise pupil(s);
measuring a parameter of said at least one eye(s) after said step of stimulating using said first physical stimulus resulting in a first measured parameter, wherein said parameter relates to a dimensional change in said at least one pupil(s) because of said physical stimulus;
waiting a period of time;
stimulating said at least one eye(s) of said living being using a second physical stimulus;
measuring said parameter of said at least one pupil(s) after said step of stimulating using said second physical stimulus resulting in a second measured parameter;
comparing said first measured parameter and said second measured parameter; and
evaluating said living being based on said comparison step.

19. The method of claim 18, wherein said step of evaluating comprises:
automatically evaluating said living being based on said comparison step using a computer, wherein said computer automatically determines whether said first measured parameter and said second measured parameter differ by enough such that said living being is determined to be intoxicated by at least one drug.

20. The method of claim 18, wherein said first physical stimulus and said second physical stimulus are the same stimulus.

21. The method of claim 18, wherein said period of time is five minutes or less.

22. The method of claim 18, wherein said first and second physical stimulus are light, and said light is adjusted according to an eye color of said living being.

23. The method of claim 18, further comprising:
automatically creating a first pupillogram based on said step of measuring said parameter of said first pupil; and
automatically creating a second pupillogram based on said step of measuring said parameter of said second pupil.

24. The method of claim 23, further comprising displaying said first pupillogram and said second pupillogram on a computer.

25. The method of claim 23, further comprising:
displaying an image of said pupil on said computer in real time; and
automatically centering said image of said pupil.

26. The method of claim 23, further comprising:
waiting a second period of time;
stimulating said either or both eye(s) of said living being using a third physical stimulus;
measuring said parameter of said either or both pupil(s) after said step of stimulating using said third physical stimulus resulting in a third measured parameter; and
comparing said third measured parameter, said second measured parameter and said first measured parameter.

27. The method of claim 26, further comprising determining that said living being is intoxicated with a drug if said third measured parameter differs from said second measured parameter or said first measured parameter.

28. The method of claim 18, wherein said first physical stimulus and said second physical stimulus is light, wherein said light is diffused.

29. A method of evaluating a living being, said method comprising the steps of:
stimulating only one eye of a living being using a physical stimulus, wherein said eye comprises a first pupil;
measuring a parameter of said first pupil of said living being after said step of stimulating said first pupil resulting in a first measured parameter, wherein said parameter relates to a dimensional change in said first pupil;
measuring said parameter of a second pupil of said living being after said step of stimulating said first pupil resulting in a second measured parameter, wherein said second pupil is not in said eye that was stimulated and wherein said parameter relates to a dimensional change in said second pupil;
comparing said first measured parameter and said second measured parameter; and
evaluating said living being based on said comparison step.

30. The method of claim 29, wherein said steps of measuring occurs automatically by a computer and a video camera.

31. The method of claim 29, further comprising:
automatically creating a first pupillogram based on said step of measuring said parameter of said first pupil; and
automatically creating a second pupillogram based on said step of measuring said parameter of said second pupil.

32. The method of claim 31, further comprising:
displaying said first pupillogram and said second pupillogram on a computer, wherein said first pupillogram is superimposed with said second pupillogram.

33. The method of claim 31, further comprising displaying an image of said pupil on said computer in real time.

34. The method of claim 29, wherein said step of evaluating comprises determining that said living being is intoxicated with a drug if said first measured parameter and said second measured parameter differ.

35. The method of claim 29, wherein said parameter comprises at least three of the following: initial pupil size, constriction latency time, parasympathetic phase amplitude, parasympathetic phase speed, parasympathetic phase gradient speed, amplitude of minimal radius value, sympathetic phase latency period, sympathetic phase speed, sympathetic period gradient speed, and sympathetic phase amplitude.

36. A method of evaluating a living being, said method comprising the steps of:
stimulating an eye of a living being using one or more physical stimulus, wherein said eye comprises an ANW;
electronically measuring a dimensional change in said ANW in response to said at least one physical stimulus;
automatically determining at least one parameter relating to said dimensional change of said ANW;
electronically storing said at least one parameter in a computer;
comparing said at least one parameter with at least one normal parameter; and
evaluating said living being based on said comparison step.

37. The method of claim 36, further comprising:
electronically measuring a dimensional change in a pupil in said eye in response to said at least one physical stimulus;
automatically determining at least one parameter relating to said dimensional change of said pupil;
electronically storing said at least one parameter relating to said dimensional change of said pupil in said computer;
comparing said at least one parameter relating to said dimensional change of said pupil with at least one normal parameter relating to said dimensional change of said pupil; and
evaluating said living being based on said comparison step of said pupil and said comparison step of said ANW.

38. The method of claim 36, further comprising:
automatically creating a first pupillogram based on said step of measuring said parameter of said ANW; and
determining a normal pupillogram.

39. The method of claim 38, wherein said normal pupillogram comprises an average pupillogram of a plurality of other normal living beings, wherein said other normal living beings have similar age and gender to said living being and said other normal living beings were not intoxicated with a drug.

40. The method of claim 36, further comprising displaying an image of said ANW on said computer in real time.

41. The method of claim 36, wherein said step of evaluating comprises determining that said living being is intoxicated with a drug if said first measured parameter and said second measured parameter differ.

42. The method of claim 36, wherein said parameter comprises at least three of the following: initial pupil size, constriction latency time, parasympathetic phase amplitude, parasympathetic phase speed, parasympathetic phase gradient speed, amplitude of minimal radius value, sympathetic phase latency period, sympathetic phase speed, sympathetic period gradient speed, and sympathetic phase amplitude.

43. The method of claim 36, wherein said step of evaluating comprises:
automatically evaluating said living being based on said comparison step using a computer.

44. A method of evaluating a living being, said method comprising the steps of:
stimulating a first and second eye of a living being using a physical stimulus, wherein said first eye comprises a first pupil and said second eye comprises a second pupil;
measuring a parameter of said first pupil of said living being after said step of stimulating said first eye resulting in a first measured parameter, wherein said first measured parameter relates to a dimensional change in said first pupil;
measuring said parameter of a second pupil of said living being after said step of stimulating said second eye resulting in a second measured parameter, wherein said second measured parameter relates to a dimensional change in said second pupil;
comparing said first measured parameter and said second measured parameter; and
evaluating said living being based on said comparison step.

45. The method of claim 44, wherein said step of evaluating comprises determining that said living being is intoxicated with a drug if said first measured parameter and said second measured parameter differ.

46. The method of claim 44, wherein said physical stimulus comprises light and said light is diffused.

47. The method of claim 44, wherein said first eye comprises a first iris and said second eye comprises a second iris, wherein said measured first parameter is wherein normalized against at least one measurement of said first iris and said measured second parameter is normalized against at least one measurement of said second iris.

48. A computer-based system for evaluating a pupil of a living being comprising:
a computer;
a living being comprising an eye, wherein said eye comprises a pupil;
a device for stimulating said eye with at least one physical stimulus;
a video camera, wherein said video camera records a plurality of images of said pupil and sends said images to said computer;
a pupillogram of said pupil, wherein said computer analyzes said images of said pupil to determine said pupillogram of said pupil over a period of time after being stimulated by said at least one physical stimulus, said pupillogram comprising information on dimensional changes in said pupil, wherein said dimensional changes are normalized against at least one measurement of said iris.

49. The system of claim 48, further comprising:
an image of said eye that is displayed on said computer;
a display of said pupillogram displayed on said computer.

50. The system of claim 48, further comprising:
at least one parameter relating to said pupillogram;
at least one normal parameter, wherein said at least one normal parameter relates to a normal pupillogram, said at least one normal pupillogram comprises information obtained from a plurality of other normal living beings that have at least one similar characteristic of said living being;
a comparison between said at least one parameter and said at least one normal parameter, wherein said computer automatically evaluates said living being based on said comparison; and
determining whether said living being is intoxicated with a drug based on said comparison step.

51. The system of claim 50, wherein said at least one similar characteristic is age or gender.

52. The system of claim 50, further comprising a display of said normal pupillogram superimposed with said display of said pupillogram.

53. The system of claim 48, further comprising an IR diode, wherein said IR diode is used to measure dimensional changes in said pupil.

54. A computer-based system for evaluating a pupil of a living being comprising:
a computer;
a living being comprising an eye, wherein said eye comprises a pupil and an iris;
a device for stimulating said eye with at least one physical stimulus;
a video camera, wherein said video camera records a plurality of images of said pupil and sends said images to said computer;
a pupillogram of said pupil, wherein said computer analyzes said images of said pupil to determine said pupillogram of said pupil over a period of time after being stimulated by said at least one physical stimulus, said pupillogram comprising information on dimensional changes in said pupil, wherein said dimensional changes are the actual measurements of said dimensional changes of said pupil.

55. The system of claim 54, further comprising at least one photodiode which measures the distance said video camera is from said eye.

56. The system of claim 54, further comprising:
at least one parameter relating to said pupillogram;
at least one normal parameter, wherein said at least one normal parameter relates to a normal pupillogram, said at least one normal pupillogram comprises information obtained from a plurality of other normal living beings that have at least one similar characteristic of said living being;
a comparison between said at least one parameter and said at least one normal parameter, wherein said computer automatically evaluates said living being based on said comparison; and
determining whether said living being is intoxicated with a drug based on said comparison step.
